(19) United States
(12) Patent Application Publication
(10) Pub. No.: US 2006/0233743 A1
(43) Pub. Date: Oct. 19, 2006

(54) Compositions and methods of therapy

(76) Inventor: Rodney William Kelly, Edinburgh (GB)

Correspondence Address: Edwin V Merkel;Nixon Peabody, Clinton Square, P O Box 31051, Rochester, NY, 14603, US

(21) Appl. No.: 10/532,291

(22) PCT Filed: Oct. 21, 2003
(86) PCT No.: PCT/GB03/04537

§ 371 Date: Mar. 13, 2006

(30) Foreign Application Priority Data
Oct. 21, 2002 (GB) 02244150

(51) Int. Cl. A61K 45/00; C12P 21/02

(52) U.S. Cl. 424/85.1; 435/69.5

(57) ABSTRACT

A method of inducing tolerance to an antigen in a patient, the method comprising administering to the patient an agent which raises the effective cAMP concentration in a monocyte cell and GMCSF or a derivative thereof. Preferably, the agent which raises the effective cAMP concentration in a monocyte cell is a prostaglandin or agonist thereof which stimulates cAMP production in a monocyte. Optionally, the antigen to which it is desired to induce tolerance, or a derivative thereof, may also be administered.

The present invention relates to therapeutic compositions, methods and uses; in particular it relates to methods for inducing tolerance to an antigen in a patient.

An organism's immunity to an antigen arises as a consequence of a first encounter with the antigen and the subsequent production of immunoglobulin molecules, for example, antibodies, capable of selectively binding that antigen. In addition, the immune response is controlled by T cells which may be antigen specific. Immunity allows the rapid recruitment, usually by stimulating an inflammatory response, of cells which can dispose of the foreign antigen. Under certain circumstances, the immune system does not produce an immune response against antigens due to a mechanism called “tolerance”. For example, an immune system can normally discriminate against foreign antigens and constituents of the organism itself, due to a mechanism whereby all B lymphocytes which could potentially produce antibodies to constituents of the organism itself (“self antigens”) are destroyed during development, thereby removing the organism's capacity to produce antibodies directed to a self antigen.

Tolerance is probably an active process. This means that peripheral tolerance is gained where an antigen is presented to a T cell in a particular tolerising environment, eg high interleukin-10 (IL-10) levels. The T cells then circulate and when they meet that specific antigen again they do not mount an immune response (anergic T cells) or they mount a quelling response (regulatory T cells). A role for regulatory T cells has been proposed in tolerance. The regulatory T cells are programmed by the environment of the antigen presenting cell to react to their cognate antigen by releasing “down-regulatory” cytokines. The first such regulatory cells described were induced by IL-10 (Groux et al., 1997, Nature 389:737-742).

Where tolerance breaks down, the organism may produce a cellular immune response (including cytotoxic T cells) to normal constituents of the organism, producing an “autoimmune disease”. Examples of autoimmune diseases include rheumatoid arthritis (RA), multiple sclerosis (MS) and systemic lupus erythematosus (SLE).

In some circumstances, even the normal response of the immune system to a foreign antigen can produce undesirable results, such as in the case of tissue or organ grafts or transplants, where the immune system of the tissue or organ recipient recognises the tissue or organ graft or transplant as foreign and acts to reject it.

One of the drawbacks of existing methods of treating immune or inflammatory conditions or diseases is the limited range of options and their therapeutic inadequacy. For example, glucocorticosteroids used for treating inflammatory respiratory disease have toxic effects in many patients, and alternatives such as cyclosporin A or interferon γ are high-risk, expensive and generally unsatisfactory.

Unexpectedly, the inventor has found that there is a marked stimulation of IL-10 in cells of the immune system when an agent which raises the effective cAMP concentration in a monocyte cell, such as a prostaglandin, and granulocyte-macrophage colony stimulating factor (GMCSF) are used in combination. Furthermore, the inventor has found that there is a synergistic effect between an agent which raises the effective cAMP concentration in a monocyte cell, such as a prostaglandin, and GMCSF on the release of IL-10 from cells of the immune system; in the presence of GMCSF the stimulation of IL-10 by both prostaglandin E (PGE) and 19-hydroxy PGE was increased strikingly, resulting in a tolerising environment. In other words, it is believed that GMCSF and an agent that raises the effective cAMP concentration in a monocyte cell, such as a prostaglandin, polarise monocytes into a phenotype characterised by increased IL-10 release. Similarly, in the presence of GMCSF the stimulation of IL-10 expression by forskolin was increased strikingly, and in a synergistic way compared to forskolin or GMCSF alone. Not only is the cell directed to a pro-tolerance phenotype but this is also accompanied by enhanced production of granulysin, an anti-microbial agent. In addition, the effects of PGE and GMCSF are prolonged and continue after the removal of these agents thus the cell is selectively differentiated.

GMCSF has an important role in granulocyte and macrophage lineage maturation. GMCSF has been proposed as both a treatment agent and a target for treatment. Recombinant human GMCSF has been used to treat some cancers and to promote haematopoietic reconstitution following bone marrow transplantation (Leukine® Package Insert Approved Text, February 1998, and Buchsel et al, (2002) Clin. J. Oncol. Nurs. 6(4): 198-205). By contrast, other recent reports describe GMCSF as being a potential target for treatment of inflammatory and immune diseases (Hamilton, (2002) Trends Immunol 23 (8):403-8) and asthma (Ritz et al, (2002) Trends Immunol 23 (8):396-402).

In diseases resulting from an aberrant or undesired immune response there is often a deficiency in IL-10. This deficiency in IL-10 may be detrimental to the development of useful T helper cells, particularly type-2 T helper cells; a preponderance of type 1 T helper cells over type 2 T helper cells is thought to be characteristic of autoimmune disease. Thus, stimulation of IL-10 production is believed to induce a tolerising environment for T cell activation. In addition, a high IL-10 environment will act on an antigen presenting cell (typically a dendritic cell) to ensure regulatory T cell formation, creating a regulatory T cell that is specific for the antigen presented.

The inventor now proposes inducing tolerance to an antigen in a patient by the use of GMCSF in combination with an agent which raises the effective cAMP concentration in a monocyte cell to induce a tolerising environment in the patient. Thus the inventor proposes that this combination induces tolerance of, or tolerance to, an antigen in a patient.

Without being bound by theory, the inventor believes that a combination of GMCSF and an agent which raises the effective cAMP concentration in a monocyte cell, such as a prostaglandin or forskolin, will also decrease IL-12 levels, which would be expected to enhance the effects of the invention. The inventor has shown that the combination of a prostaglandin and GMCSF increases the expression of both IL-10 and COX-2, and that the combination of forskolin and GMCSF synergistically increases the level of IL-10 in a monocyte cell. The decrease in IL-12 levels may therefore arise through the direct inhibition of IL-12 by IL-10 (Harizi et al., 2002) or through an IL-10 independent pathway that depends on COX-2 induction (Schwacha et al., 2002).

The combination of a GMCSF and an agent which raises the effective cAMP concentration in a monocyte cell, such as a prostaglandin or agonist thereof is also considered by the inventor to achieve the desirable effect of reducing the amount of agent, such as prostaglandin or agonist, or GMCSF required to achieve a useful degree of therapeutic benefit, hence reducing the side effects of administration of the prostaglandin or agonist thereof or the GMCSF.

As far as the inventor is aware, there has never been any suggestion that a combination of an agent which raises the effective cAMP concentration in a monocyte cell, such as a prostaglandin or an agonist thereof, and GMCSF could be used to stimulate IL-10 production, and there has been no suggestion of a treatment using this combination to stimulate IL-10. Furthermore, there has never been any suggestion that this combination could be used to induce a tolerising environment for T cell activation, or to induce tolerance to an antigen in a patient. Moreover, there has been no mention of an anti-microbial effect accompanying tolerance induction or indeed of an anti-microbial effect from monocytes in any application.

The inventor further proposes inducing tolerance to a specific antigen in a patient by the use of an agent which raises the effective cAMP concentration in a monocyte cell, such as a prostaglandin or agonist thereof, in combination with GMCSF and the specific antigen to which it is desired to induce tolerance, or a derivative thereof.

As far as the inventor is aware, there has never been any suggestion that administering an agent which raises the effective concentration of cAMP in a monocyte cell, such as a prostaglandin or agonist thereof or forskolin, an antigen or derivative thereof, and GMCSF to a patient could be used to induce tolerance to that antigen in the patient.

The inventor has also shown that PGE and GMCSF reduce levels of participants in antigen presentation such as class II transactivator (CIITA) and MHC class II (as shown in Example 1). This change in phenotype is accompanied by enhanced expression of granulysin which has antimicrobial, including antiviral, properties (Krensky 2000) and is normally thought of as a product of activated T cells that mediates antiviral activity that lyses infected cells (Hata et al. 2001; Ochoa et al. 2001; Smyth et al. 2001). The increased expression of granulysin is believed to be an important consequence of the present invention, as the increase in innate defence molecules may compensate for the compromise of the adaptive immune system that accompanies tolerance induction.

In addition, the inventor has shown that a combination of PGE and GMCSF increases the expression of COX-2, CD86, CD14. COX-2 is believed to be involved in maintaining the tolerant phenotype after removal of the prostaglandin and GMCSF (as is shown in Examples 2 and 3), and both CD14 and CD86 are differentiation markers and are evidence of a more differentiated state. Furthermore, the inventor has shown that differentiation with forskolin and GMCSF does not appreciably raise TNFα in monocyte cells (TNFα is a pro-inflammatory and anti-tolerogenic agent).

The listing or discussion of a prior-published document in this specification should not necessarily be taken as an acknowledgement that the document is part of the state of the art or is common general knowledge.

A first aspect of the invention provides a composition comprising an agent which raises the effective concentration of cAMP in a monocyte cell and GMCSF or a derivative thereof.

The agent which raises the effective cAMP concentration in a monocyte cell may do so in several distinct but related biochemical ways. Thus, the agent may be one which increases the production of cAMP, for example by the stimulation of receptors which are linked to the production of cAMP. Such agents include prostaglandins and agonists thereof which are described in more detail below. Cholera toxin can also be used to increase cAMP levels intracellularly as has been described in Braun et al (1999) J. Exp. Med. 189, 541-552 and there is also evidence that it may increase antigen transport across the epithelium which may be desirable. Similarly, β-adrenergic agents, which raise cAMP levels within a cell via the β-adrenergic receptor, may be used. Such β-adrenergic agents are well known in the art, such as in the treatment of asthma. Suitable β-adrenergic agents include isoproterenol.

The agent may be one which inhibits the breakdown of cAMP and thus may be a cAMP phosphodiesterase inhibitor, which are described in more detail below. The agent may be one which inhibits the export of cAMP from the cell. Export of cAMP from the cell is via a specific transporter (typically the multidrug resistance protein, MRP-4) which may be blocked with, for example, probenicid (a drug currently used for gout) or progesterone or agonists or antagonists thereof, such as medroxyprogesterone acetate or RU 486, which also appears to have an inhibitory effect on the cAMP transporter.

The agent may also be a compound which mimics the effects of cAMP in the cell in relation to generating a pro-tolerant state but which may be less susceptible to degradation or export. Such compounds, when present in the cell can be considered to raise the effective cAMP concentration. Such compounds include Sp-adenosine 3′,5′-cyclic monophosphorothioate and 8-bromoadenosine 3′,5′-cyclic monophosphate and dibutyryl cAMP. That sufficient of these compounds have been administered may be assessed by determining that there has been an elevation in IL-10 expression in monocyte cells. Preferably, the agent when used at a concentration which gives a maximal response elevates IL-10 expression at least 1.2-fold, or 1.5-fold, or 2-fold, or 5-fold, or 10-fold. Typically, from around 1 to 100 μmol of the cAMP analogues may be administered to the patient.

Forskolin is 7β-Acetoxy-8,13-epoxy-1α,6β,9α-trihydroxylabd-14-en-11-one 7β-Acetoxy-1α,6β,9α-trihydroxy-8,13-epoxy-labd-14-en-11-one. It is also called Coleonol and Colforsin and has a Mᵣ of 410. It is a cell-permeable diterpenoid that possesses anti-hypertensive, positive inotropic and adenyl cyclase activating properties. Many of its biological effects are due to its activation of adenylate cyclase and the resulting increase in intracellular cAMP concentration. Forskolin affects calcium currents and inhibits MAP kinase. Colforsin is used as daropate (see Ann Thoracic Surgery (2001) 71, 1931-1938). It may be administered as the hydrochloride to ensure water solubility but it may also be used as the free base which may be able to more readily penetrate cell membranes.

Sp-Adenosine 3′,5′-cyclic monophosphorothioate (SpcAMP) has a Mᵣ of 446 and is the Sp-diastereomer of adenosine-3′,5′-cyclic monophosphothioate. It is a potent, membrane-permeable activator of cAMP dependent protein kinase I and II that mimics the effects of cAMP as a second messenger in numerous systems while being resistant to cyclic nucleotide phosphodiesterases. It exhibits greater specificity and affinity than forskolin and cAMP analogues such as dibutyryl-cAMP.

8-Bromoadenosine 3′,5′-cyclic monophosphate (8-BrcAMP) has a Mᵣ of 430. It is a cell-permeable cAMP analogue having greater resistance to hydrolysis by phosphodiesterases than cAMP. It activates protein kinase A.

Cholera toxin has a Mᵣ of around 100,000. It is a toxin consisting of an A subunit (27 kDa) surrounded by five B subunits (approximately 12 kDa each), which attach the toxin to ganglioside GM1 on the cell surface. The A subunit catalyzes ADP-ribosylation of the α-subunit of the stimulatory G protein (Gαs) reducing GTPase activity and activating the α-subunit. This activation of Gαs leads to an increase in the activity of adenylate cyclase resulting in increased levels of cAMP. It also ADP-ribosylates transducin in the eye rod outer segments, inactivating its GTPase activity. Cholera toxin has also been reported to ADP-ribosylate tubulin. It has been shown to be a potent mucosal vaccine adjuvant, inducing T helper cell type 2 responses by inhibiting the production of interleukin-12 (Braun et al (1999) supra). Although fragments of cholera toxin which are able to increase cAMP levels in monocytes may be used, it is preferred that complete cholera toxin is used.

Since cholera toxin may, under some conditions, induce anaphylaxis (oversensitization), it is less preferred.

It is likely that SpcAMP and 8-BrcAMP, and possibly forskolin, inhibit the cAMP export pump and this may contribute to their ability for raising the effective cAMP concentration.

It is convenient to measure the effective cAMP concentration in monocyte cells (ie by assessing the effect of the agent on monocyte cells). A preferred monocyte cell is the well known human monocyte cell line U937. It will be appreciated that the agents will also raise the effective cAMP concentration in other monocyte and monocyte-related cells such as macrophages, and that the utility in the context of the invention may be due to the effect on these cells. As noted above, whether or not there is a sufficient amount of cAMP analogues can be determined by measuring IL-10 in monocyte cells. Preferably, the agent when used at a concentration which gives a maximal response raises the cAMP concentration at least 1.2-fold, or 1.5-fold or 2-fold or 5-fold or 10-fold.

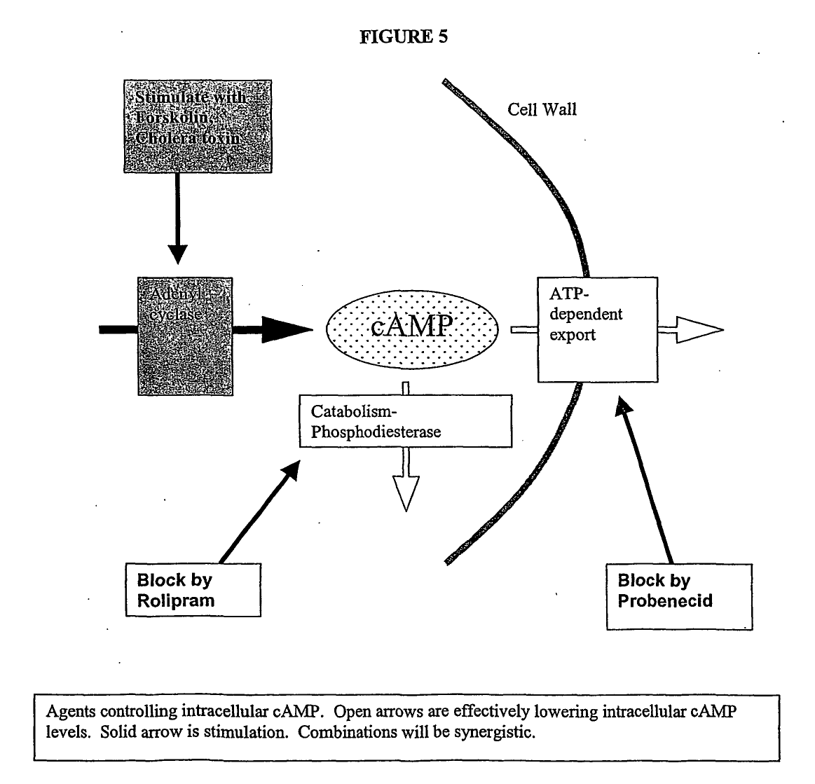

FIG. 5 shows diagrammatically various places of intervention in or on a cell which lead to raising cAMP levels.

It is preferred that the agent which raises the effective cAMP concentration in a monocyte cell is a prostaglandin.

It is preferred for this and all other aspects of the invention that the prostaglandin or agonist thereof stimulates cAMP production in a monocyte.

The prostaglandin or agonist thereof may be any suitable prostaglandin or agonist thereof that stimulates cAMP production in a monocyte, and which particularly in the presence of GMCSF causes monocytes to express IL-10. Prostaglandins or agonists thereof that are suitable for use in the present invention may readily be determined by a person of skill in the art. Methods for assessing cAMP production in monocytes may be found in Burzyn et al., (2000) and in Example 3, and methods for detecting IL-10 expression in and release from monocytes include those in Examples 1 and 3.

By “prostaglandin or agonist” we mean any compound which acts as a prostaglandin agonist on a prostaglandin receptor. The prostaglandin agonist may be, but need not be, a prostanoid. Typically, the prostaglandin or agonist is one which binds the EP2 or EP4 receptor. The prostaglandin may be a PGE, a PGD or a PGI, or an agonist thereof. Preferably, the prostaglandin is a PGE or an agonist thereof. It is appreciated that PGI may be too unstable to be useful as a pharmacological agent, however PGI₂ and stable analogues of PGI may be suitable. Preferably, the prostaglandin is not a PGF or an agonist thereof.

It is preferred that the prostaglandin or agonist thereof is PGE₂ or a synthetic analogue thereof. Synthetic analogues include those modified at position 15 or 16 by the addition of a methyl group or those where the hydroxyl has been transposed from position 15 to position 16. Preferred examples of analogues of prostaglandin include Butaprost (an EP2 receptor agonist) and 11-deoxy PGE1 (an EP4 receptor agonist) and 19-hydroxy PGE. For the avoidance of doubt, the term “prostaglandin” includes naturally-occurring prostaglandins as well as synthetic prostaglandin analogues.

Suitable prostaglandins or agonists thereof include dinoprostone (sold as Propess by Ferring in Europe and Forest in the USA; sold as Prostin E2 by Pharmacia), gemeprost (sold by Farillon), misoprostol (which is sold as Cytotec by Searle and Pharmacia), alprostadil (which is sold as Caverject by Pharmacia and Viridal by Schwarz and MUSE by AstraZeneca) and limaprost.

Misoprostol is a PGE analogue which has EP2 and EP3 agonist effects. Its chemical structure is (±) methyl 11α,16-dihydroxy-16-methyl-9-oxoprost-13-enoate.

An example of a non-prostanoid compound which acts as a prostaglandin agonist is AH23848, an EP4 receptor agonist.

EP2 agonists which may be useful in the practise of the invention include AH13205.

Suitable prostaglandins also include 19-hydroxy PGE1 and 19-hydroxy PGE2. Prostaglandin E agonists are described in EP 1 097 922 and EP 1 114 816, incorporated herein by reference.

Suitable prostaglandins or agonists thereof may also include any of the 19-hydroxy prostaglandin analogues described in U.S. Pat. No. 4,127,612, incorporated herein by reference.

It is preferred that the prostaglandin is prostaglandin E₂ (PGE₂) or 19-hydroxy PGE. Prostaglandins and agonists thereof, including PGE₂, are commercially available, for example from Pharmacia and Upjohn as Prostin E2.

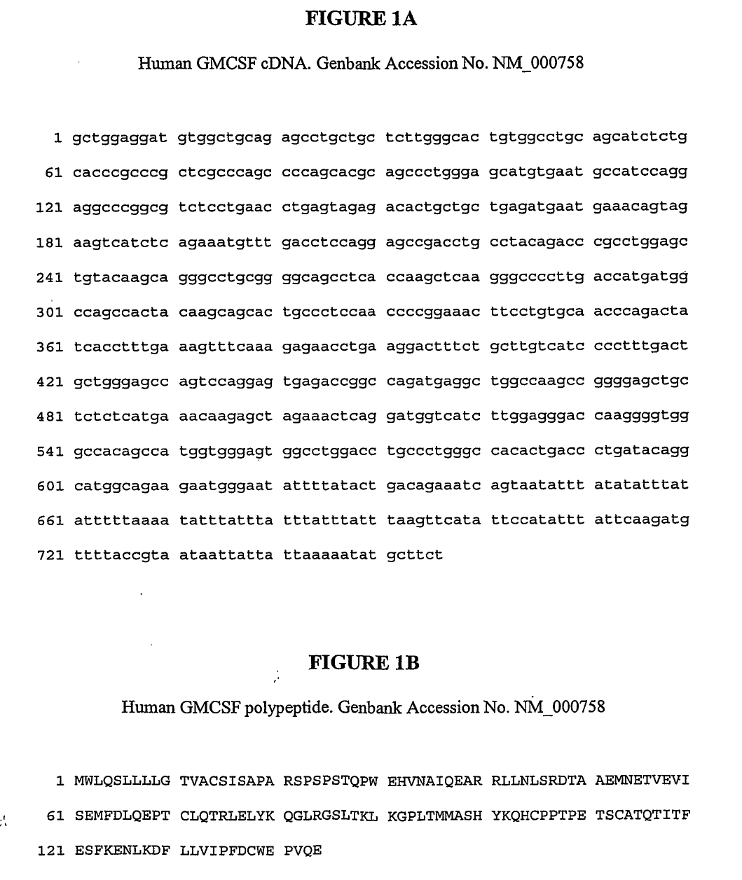

By “GMCSF” we include the gene product of the human GMCSF gene and naturally occurring variants thereof. The nucleotide and the amino acid sequence of human GMCSF is found in Genbank Accession No. NM_{—}000758, and in FIG. 1. Some naturally occurring variants of GMCSF are also listed in NM_{—}000758. GMCSF is also known as colony stimulating factor 2 (CSF2).

The invention includes the use of derivatives of GMCSF that retain the biological activity of wild-type GMCSF, ie that stimulate the production of granulocytes and macrophages from their progenitor cells, and which in the presence of prostaglandin E cause monocytes to express IL-10.

By “derivative” of GMCSF we include a fragment, fusion or modification or analogue thereof, or a fusion or modification of a fragment thereof.

By “fragment” of GMCSF we mean any portion of the glycoprotein that stimulates the production of granulocytes and macrophages from their progenitor cells and which in the presence of prostaglandin E causes monocytes to express IL-10. Typically, the fragment has at least 30% of the activity of full length GMCSF. It is more preferred if the fragment has at least 50%, preferably at least 70% and more preferably at least 90% of the activity of full length GMCSF. Most preferably, the fragment has 100% or more of the activity of full length GMCSF.

The derivatives may be made using protein chemistry techniques for example using partial proteolysis (either exolytically or endolytically), or by de novo synthesis. Alternatively, the derivatives may be made by recombinant DNA technology. Suitable techniques for cloning, manipulation, modification and expression of nucleic acids, and purification of expressed proteins, are well known in the art and are described for example in Sambrook et al (2001) “Molecular Cloning, a Laboratory Manual”, 3^{rd} edition, Sambrook et al (eds), Cold Spring Harbor Laboratory Press, Cold Spring Harbor, N.Y., USA, incorporated herein by reference.

The invention also includes modifications of full length GMCSF, or a fragment thereof, that stimulate the production of granulocytes and macrophages from their progenitor cells and which in the presence of prostaglandin E cause monocytes to express IL-10.

Such modifications include deglycosylating the glycoprotein either fully or partially. Other modifications include full length GMCSF, or a fragment thereof, having a different glycosylation pattern from that found in naturally occurring human GMCSF.

Other modifications of full length GMCSF, or a fragment thereof, include amino acid insertions, deletions and substitutions, either conservative or non-conservative, at one or more positions. Such modifications may be called analogues of GMCSF. By “conservative substitutions” is intended combinations such as Gly, Ala; Val, Ile, Leu; Asp, Glu; Asn, Gln; Ser, Thr; Lys, Arg; and Phe, Tyr. Such modifications may be made using the methods of protein engineering and site-directed mutagenesis, as described in Sambrook et al 2001, supra. Preferably, the modified GMCSF or modified GMCSF fragment retains at least 30% of the activity of full length GMCSF. It is more preferred if the modified GMCSF or GMCSF derivative has at least 50%, preferably at least 70% and more preferably at least 90% of the activity of full length GMCSF. Most preferably, the modified GMCSF or modified GMCSF fragment has 100% or more of the activity of full length GMCSF.

The invention also includes a fusion of full length GMCSF, or a fragment thereof, to another compound. Preferably, the fusion retains at least 30% of the activity of full length GMCSF. It is more preferred if the fusion has at least 50%, preferably at least 70% and more preferably at least 90% of the activity of full length GMCSF. Most preferably, the fusion has 100% or more of the activity of full length GMCSF.

GMCSF and analogues thereof are described in the following publications, each of which are incorporated herein by reference: U.S. Pat. No. 5,229,496 (Deeley et al.); U.S. Pat. No. 5,391,485 (Deeley et al.); U.S. Pat. No. 5,393,870 (Deeley et al.); U.S. Pat. No. 5,602,007 (Dunn et al.); Wong et al, “Human GM-CSF: molecular cloning of the complementary DNA and purification of the natural and recombinant proteins”, Science 228 (4701), 810-815 (1985); Lee et al, “Isolation of cDNA for a human granulocyte-macrophage colony-stimulating factor by functional expression in mammalian cells”, Proc. Natl. Acad. Sci. U.S.A. 82 (13), 4360-4364 (1985); Cantrell et al, “Cloning, sequence, and expression of a human granulocyte/macrophage colony-stimulating factor”, Proc. Natl. Acad. Sci. U.S.A. 82 (18), 6250-6254 (1985); and Miyatake et al, “Structure of the chromosomal gene for granulocyte-macrophage colony stimulating factor: comparison of the mouse and human genes”, EMBO J. 4 (10), 2561-2568 (1985).

While it is preferred that GMCSF is human GMCSF as defined above, by GMCSF we also include GMCSF from other species. However, it is appreciated that for applications in which GMCSF is administered to a subject, the GMCSF is preferably from the same species as the subject. Thus if the GMCSF is to be administered to a human subject, the GMCSF is preferably human GMCSF.

Suitable GMCSF for the practice of this invention can be obtained from Peprotech EC Ltd., 29 Margravine Road, London, W6 8LL, catalogue number 300-03.

A preferred GMCSF for the practice of this invention is sargramostim, the proper name for yeast-derived recombinant human GMCSF, sold under the trade name Leukine® produced by Immunex, Inc. Leukine® is a recombinant human GMCSF produced in a S. cerevisiae expression system. Leukine® is a glycoprotein of 127 amino acids characterised by 3 primary molecular species having molecular masses of 19,500, 16,800 and 15,500 Daltons. The amino acid sequence of Leukine® differs from natural human GMCSF by a substitution of leucine at position 23, and the carbohydrate moiety may be different from the native protein. Leukine® is suitable for subcutaneous or intravenous administration (Leukine® Package Insert Approved Text, February 1998).

Unless the context indicates otherwise, wherever the term “GMCSF” is used, a derivative as herein defined is included.

In a preferred embodiment, the composition is a pharmaceutical composition comprising an agent which raises the effect cAMP concentration in a monocyte cell, such as a prostaglandin or agonist thereof, and GMCSF and a pharmaceutically acceptable carrier, diluent or excipient (including combinations thereof).

The carrier, diluent or excipient must be “acceptable” in the sense of being compatible with the composition of the invention and not deleterious to the recipients thereof. Typically, the carriers will be water or saline which will be sterile and pyrogen free.

Acceptable carriers or diluents for therapeutic use are well known in the pharmaceutical art, and are described, for example, in Remington's Pharmaceutical Sciences, Mack Publishing Co. (A. R. Gennaro edit. 1985). The choice of pharmaceutical carrier, excipient or diluent can be selected with regard to the intended route of administration and standard pharmaceutical practice. The pharmaceutical compositions may comprise as, or in addition to, the carrier, excipient or diluent any suitable binder(s), lubricant(s), suspending agent(s), coating agent(s), or solubilising agent(s).

Preservatives, stabilizers, dyes and even flavoring agents may be provided in the pharmaceutical composition. Examples of preservatives include sodium benzoate, sorbic acid and esters of p-hydroxybenzoic acid. Antioxidants and suspending agents may be also used.

In an embodiment, the composition further comprises an antigen to which it is desired to induce tolerance in a subject, or a derivative thereof. Details of preferred antigens for use in the practice of the present invention are provided below. Thus, the invention includes a composition comprising an agent which raises the effective cAMP concentration in a monocyte cell, such as a prostaglandin or an agonist thereof, GMCSF or a derivative thereof, and an antigen to which it is desired to induce tolerance, or a derivative thereof. The invention also includes a pharmaceutical composition comprising an agent which raises the effective cAMP concentration in a monocyte cell, such as a prostaglandin or an agonist thereof, GMCSF or a derivative thereof, and an antigen to which it is desired to induce tolerance, or a derivative thereof, and a pharmaceutically acceptable carrier, diluent or excipient (including combinations thereof).

It is appreciated that to induce tolerance to an antigen, a derivative of the antigen may be administered to the patient, and not the antigen itself. By “derivative” of an antigen we include any portion of the antigen which can be presented by a class I or a class II MHC molecule for example on an antigen presenting cell (APC), and which induces tolerance to the antigen itself. Typically the derivative of the antigen is also recognised by a T cell when presented, for example via a T cell receptor.

When the antigen is a protein, a derivative of the antigen is typically a peptide fragment of the antigen consisting of a contiguous sequence of amino acids of the antigen capable of MHC binding. Preferably, the fragment is between 6 and 100 amino acids in length. More preferably, the fragment is between 6 and 50 amino acids in length. Most preferably, the fragment is six, or seven, or eight, or nine, or ten, or eleven, or twelve, or thirteen, or fourteen, or fifteen, or sixteen, or seventeen, or eighteen, or nineteen, or twenty, or twenty-one, or twenty-two, or twenty-three, or twenty-four or twenty-five amino acids in length.

A derivative of the antigen may include a fusion of the antigen, or a fusion of a fragment of the antigen, to another compound, and which can be recognised by either a class I or a class II MHC molecule when presented, and which induces tolerance to the antigen itself. Typically, the fusion is one which can be processed by an APC so as to present a portion which is able to induce tolerance to the antigen itself.

Unless the context indicates otherwise, wherever the term “antigen” is used in the context of an antigen, a derivative as herein defined is included.

Without being bound by theory, the inventor believes that the agent which raises the effective cAMP concentration in a monocyte, such as a prostaglandin or agonist thereof, and GMCSF induce tolerance to an antigen by synergistically stimulating IL-10 production in, and secretion from, monocytes. The effect of the invention may be further increased by using a chemotactic agent that induces monocytes into the tissue to which the agent, such as prostaglandin, and GMCSF and, optionally the antigen, are administered, and the newly arrived monocytes are then directed into the tolerant phenotype.

Thus in an embodiment, the composition may further comprise a monocyte-attracting chemotactic agent. Suitable chemotactic agents for the practice of this invention include MIP-1α and MCP-1, which can be obtained from Peprotech EC Ltd., 29 Margravine Road, London, W6 8LL, catalogue number 300-04. Other suitable chemotactic agents are described in U.S. Pat. No. 5,908,829 to Kelly, incorporated herein by reference.

The inventor further believes that it may be beneficial to include a phosphodiesterase (PDE) inhibitor in the composition. The principal receptors for prostaglandin E2 (PGE2) are the EP2 and EP4 sub-types; however, other receptor sub-types exist (namely EP1 and EP3). EP2 and EP4 receptors couple with adenylcyclase and use elevated cAMP as the messenger system. The levels of cAMP in tissue are governed both by its synthesis and by its catabolism by PDEs which can be blocked by specific PDE inhibitors. Thus, the inventor believes that the effect of a prostaglandin or agonist thereof (such as PGE) acting on its EP2 and EP4 receptors is to stimulate cAMP, and the addition of the PDE inhibitor provides a synergistic action on monocytes and macrophages resulting in a reduction in the immune and/or inflammatory response which is greater than the effect of the sum of the same amount of the prostaglandin or agonist thereof and GMCSF, or PDE inhibitor administered alone.

Moreover, the inventor has previously found that the combination of a prostaglandin and a PDE inhibitor markedly stimulate IL-10 and inhibit IL-12 expression in, and secretion from, cells of the immune system, resulting in a tolerising environment.

Thus in an embodiment, the composition may further comprise a PDE inhibitor.

The PDE inhibitor may be any suitable PDE inhibitor. Preferably, the PDE inhibitor is one which inhibits a PDE which is active in cAMP breakdown. The PDEs which are known to be active in cAMP breakdown are those of the types IV, VII and VIII. Preferably, the PDE inhibitors are selective for type IV or VII or VIII.

Most preferably, the PDE inhibitors are selective for type IV PDE. By “selective” we mean that the inhibitor inhibits the particular type of PDE inhibitor for which it is selective, more potently than another type. Preferably, the type IV selective inhibitor is at least 2 times more potent an inhibitor of type IV PDE than another PDE type. More preferably, the type IV selective inhibitor is at least 5 times, 10 times, 20 times, 30, times 40 times, 50 times, 100 times, 200 times, 500 times or 1000 times more potent an inhibitor of type IV PDE than another PDE type.

Typically, the selective inhibitor is around 5 to 50 times more potent an inhibitor of the selected PDE type than another PDE type. Typically, the selective inhibitor is 5 to 50 times more potent an inhibitor of the selected PDE type than an inhibitor that is considered to be non-selective such as theophylline. Thus, theophylline is 30 times less effective than rolipram.

Preferably, selective inhibition is determined by a comparison of IC₅₀ levels (Dousa (1999) Kidney International 55: 29-62).

Non-specific PDE inhibitors include caffeine, theophylline, 3-isobutyl-1-methylxanthine (IBMX) and pentoxifylline (3,7-dihydro-3,7-dimethyl-1-(5-oxohexyl)-1H-purine-2,6-dione), although caffeine is not as active as the others and so is less preferred. The IC₅₀ value for IBMX is 2-50 μM.

U.S. Pat. No. 6,127,378, incorporated herein by reference, discloses phenanthridines substituted in the 6 position that are described as selective PDE inhibitors (mainly of type IV), that may be suitable for use in the methods of the invention.

Specific (or selective) type IV PDE inhibitors include rolipram (4-[3-cyclopentyloxy-4-methoxyphenyl]-2-pyrrolidinone) and Ro-20-1724 (4-[3-butoxy-4-methoxybenzyl]-2-imidazolidinone). The IC₅₀ for rolipram is 800 nM, and the IC₅₀ for Ro-20-1724 is 2 μM.

Another suitable PDE type IV selective inhibitor is denbufylline (1,3-di-n-butyl-7-(2-oxopropyl)-xanthine).

CP 80 633 (Hanifin et al (1996) J. Invest. Dermatol. 107, 51-56), CP 102 995 and CP 76 593 are also all potent type IV inhibitors (available from Central Research Division, Pfizer Inc, Groton, Conn.).

Other high affinity type IV selective PDE inhibitors include CPD 840, RP 73401, and RS 33793 (Dousa, 1999). The high affinity type IV selective PDE inhibitors have a Kᵢ of approximately 1 nM while the lower affinity inhibitors have a Kᵢ of about 1 μM.

The disclosures in Dousa (1999); Müller et al (1996, Trends Pharmacol. Sci. 17: 294-298); Palfreyman & Souness (1996, Prog Med Chem 33: 1-52); Stafford & Feldman (1996, Annual Reports in Medicinal Chemistry (vol 31) pp 71-80; Ed. Bristol, Academic Press, NY, USA); and Teixeira et al (1997, Trends Pharmacol. Sci. 18: 164-171) relating to type IV PDE selective inhibitors are incorporated herein by reference.

Typically, when a type IV PDE-selective inhibitor is administered orally, around 1 to 30 mg is used. Thus, a typical oral dose of rolipram or denbufylline is 1 mg or 5 mg or 10 mg or 30 mg. When a non-selective PDE inhibitor is used, such as theophylline, and it is administered orally, the dose is between 5 and 50 mg, such as 5 or 10 or 20 or 30 or 40 or 50 mg.

When the composition includes progesterone, it is preferred if the dose of progesterone is sufficient to provide levels of between 100 nM and 50 μM.

Preferred combinations, together with GMCSF, are:

PGE

PGE+Rolipram

PGE+probenecid

PGE+Rolipram+probenecid

Forskolin

Forskolin+Rolipram

Forskolin+Rolipram+probenecid

8-Bromo cAMP+probenecid

8-Bromo cAMP+Rolipram+probenecid

Sp-Adenosine 3,5-cyclic monophosphothioate (SpcAMP)

SpcAMP+probenecid

SpcAMP+Rolipram+probenecid

Cholera toxin

Cholera toxin+probenecid

The inventor believes that these (and other) combinations may act synergistically to desirably raise the effect cAMP levels in monocyte cells. It will also be appreciated that by manipulating all the metabolic points for cAMP (see FIG. 5), a lower dose of the components of the mixture would be possible in order to give the same effect compared to a single component alone.

A second aspect of the invention provides a method of inducing tolerance to an antigen in a patient, the method comprising administering to the patient an agent which raises the effective cAMP concentration in a monocyte cell and GMCSF or a derivative thereof.

By inducing tolerance to an antigen we include the meaning that the immune system of the patient may become tolerant of an antigen where it was intolerant before, or the immune system may mount a reduced response or no response at all (ie, an undetectable response) to the antigen.

The invention also provides a method of inducing tolerance to an antigen in a patient, the method comprising administering to the patient an agent which raises the effective cAMP in a monocyte cell, GMCSF or a derivative thereof, and the antigen or derivative thereof.

The antigen or derivative thereof administered is typically the antigen to which it is desired to induce tolerance or a derivative thereof.

The agent which raises the effective cAMP concentration in a monocyte cell, GMCSF or derivative thereof, and the antigen or derivative thereof, are preferably as described above with respect to the first aspect of the invention.

It is particularly preferred if the agent is a prostaglandin or agonist thereof.

The invention thus includes a method of inducing tolerance to an antigen in a patient comprising administering to the patient a composition as defined in the first aspect of the invention.

The method may further comprise administering a monocyte chemotactic agent, such as one described above.

It will be appreciated that the amount of agent which raises the effective cAMP concentration in a monocyte cell in combination with GMCSF or derivative thereof, will be sufficient when administered to the patient to have the desired therapeutic effect of inducing a tolerant state and/or suppressing the immune system or an inflammatory response in the patient.

The method may comprise administering a PDE inhibitor, such as one described above.

The invention includes suppressing the immune system or an inflammatory response in a patient. By “suppressing” we include the meaning that the immune system or the inflammatory response is altered such that, in the case of an inflammatory response, a reduced inflammatory response to a stimulus is obtained, or an inflammatory response is avoided to the extent that a response is undetectable.

Accordingly, the invention includes inducing tolerance to an antigen in a patient to treat an aberrant or undesired immune or inflammatory response in the patient. This may be particularly useful in the treatment of diseases or conditions where there is an undesirable inflammatory response or immune response. The invention therefore includes a method of suppressing an immune response or an inflammatory response in a patient, the method comprising administering to the patient an agent which raises the effective cAMP concentration in a monocyte cell and GMCSF. The invention further includes a method of suppressing an immune response or an inflammatory response in a patient comprising administering to the patient an agent which raises the effective cAMP concentration in a monocyte cell, GMCSF, and an antigen to which it is desired to induce tolerance or a derivative thereof.

By “aberrant or undesired immune or inflammatory response” we include diseases or conditions which cause the presence of visible or measurable inflammation within a tissue in an individual or patient. For example, the tissue that forms part of an allograft or the tissues of a host having received an allograft, or the central nervous system of an individual with MS, or insulitis in a patient with type 1 diabetes, or swollen joints in a patient with rheumatoid arthritis.

The invention includes a method of inducing tolerance to an antigen in a patient thereby suppressing an aberrant or undesired immune or inflammatory response in the patient, such as a response related to transplant rejection.

Therefore, in one embodiment, the invention includes the treatment of a disease or condition associated with transplant rejection such as graft versus host disease or host versus graft disease, for example in organ or skin transplants. In these cases, an inhibition or dampening of an immune or inflammatory response may be required. Thus, the invention includes the combating of transplant rejection.

The invention includes a method of combating transplant rejection, or a disease or condition associated with transplant rejection, in a patient, the method comprising administering to the patient an agent which raises the effective cAMP concentration in a monocyte cell, GMCSF, and optionally an antigen to which it is desired to induce tolerance or a derivative thereof.

If the disease or condition associated with transplant rejection is graft versus host disease, typically the antigen is a host antigen, ie an antigen present in the transplant recipient. Alternatively, if the disease or condition associated with transplant rejection is host versus graft disease, typically the antigen is one which is present on the transplanted organ or material. In these cases, an inhibition or dampening of an immune or inflammatory response associated with an increase in T regulatory cells specific for antigens in the transplant may be required. Preferably the antigen is a class I MHC molecule. Most preferably, the MHC molecule is HLA-A2.

Diseases or conditions where there is an aberrant or undesired immune or inflammatory response may also include allergies, wherein the undesired response is an allergic response. In such a condition or disease, the antigen to which tolerance is induced would be an allergen.

Thus the methods of the invention may be particularly useful in the treatment of an allergic condition or disease where there is an undesirable allergic inflammatory or immune response.

Thus in another embodiment, the invention includes a method of treating, preventing or suppressing an allergic response in a patient, the method comprising administering to the patient an agent which raises the effective cAMP concentration in a monocyte cell and GMCSF. Optionally, an antigen to which it is desired to induce tolerance, or a derivative thereof, is also administered to the patient. Typically, in an allergic condition or disease, the antigen to which tolerance is induced would be an allergen.

The allergy may be any allergy such as allergy to cat dander, house dust mite, grass or tree pollens, fungi, moulds, foods, stinging insects and so on.

In one preferred embodiment, the allergic condition or disease is allergic asthma. Preferably, the agent which raises the effective cAMP concentration in a monocyte cell and/or the GMCSF and/or the antigen or derivative thereof, are administered to the lungs or bronchial tree via an aerosol. It is preferred if the agent is a prostaglandin or agonist thereof. This embodiment may be particularly advantageous as some 19-hydroxy prostaglandin analogues have been reported to function as bronchodilators, such as those described in U.S. Pat. No. 4,127,612, incorporated herein by reference. The reason why prostaglandins are not widely used in the treatment of asthma is that they make the patient cough. Administration of GMCSF would allow the prostaglandin to be administered at a lower concentration, thus providing the therapeutic benefits while minimising the side-effects.

Thus the invention includes the use of a 19-hydroxy PGE, GMCSF, and optionally an allergen to which it is desired to induce tolerance for treatment by inhalation of allergic asthma, or a derivative thereof.

In one preferred embodiment, if the disease or condition is an allergic disease or condition, such as allergic asthma, the antigen may be a mite allergen, a dust allergen, or a mammalian allergen such as a cat or a dog or a horse allergen, preferably a cat allergen.

In other embodiments, the antigen (allergen) may be any of the following: Fel d 1 (the feline skin and salivary gland allergen of the domestic cat Felis domesticus—the amino acid sequence of which is disclosed in WO 91/06571); Der p I, Der p II, Der fI or Der fII (the major protein allergens from the house dust mite dermatophagoides—amino acid sequences disclosed in WO 94/24281); and allergens present in any of the following: grass, tree and weed (including ragweed) pollens; fungi and moulds; foods eg fish, shellfish, crab lobster, peanuts, nuts, wheat gluten, eggs and milk; stinging insects eg bee, wasp and hornet and the chirnomidae (non-biting midges); spiders and mites, including the house dust mite; allergens found in the dander, urine, saliva, blood or other bodily fluid of mammals such as cat, dog, cows, pigs, sheep, horse, rabbit, rat, guinea pig, mouse and gerbil; airborne particulates in general; latex; and protein detergent additives.

The antigen (allergen) may also be an insect antigen, selected from the group of insects comprising: housefly, fruit fly, sheep blow fly, screw worm fly, grain weevil, silkworm, honeybee, non-biting midge larvae, bee moth larvae, mealworm, cockroach and larvae of Tenibrio molitor beetle.

In still another embodiment, the invention includes a method of treating an autoimmune disease in a patient, the method comprising administering to the patient an agent which raises the effective cAMP concentration in a monocyte cell and GMCSF. The treatment of an autoimmune disease may involve inducing tolerance to a self-antigen against which there is an undesired immune response.

Autoimmune diseases that may be treated using the methods of the present invention include primary myxoedema, thyrotoxicosis, pernicious anaemia, autoimmune atrophic gastris, Addison's disease, insulin-dependent diabetes mellitus (IDDM), Goodpasture's syndrome, myasthenia gravis, sympathetic ophthalmia, MS, autoimmune haemolytic anaemia, idiopathic leucopenia, ulcerative colitis, dermatomyositis, scleroderma, mixed connective tissue disease, rheumatoid arthritis, irritable bowel syndrome, SLE, Hashimoto's disease, thyroiditis, Behcet's disease, coeliac disease/dermatitis herpetiformis, and demyelinating disease.

For example, for treating arthritis, the agent which raises the effective cAMP concentration in a monocyte cell and GMCSF or derivative thereof may be administered to synovial fluid to ensure an immunologically tolerant ambience.

In an embodiment, the method of treating an autoimmune disease in a patient, further comprises administering to the patient an antigen to which it is desired to induce tolerance, or a derivative thereof. Typically, the antigen is a self-antigen against which there is an undesired immune response.

Preferably, if the disease or condition is pernicious anaemia, the antigen may be vitamin B₁₂.

Preferably, if the disease or condition is Addison's disease, the antigen may be adrenal antigen.

Preferably, if the disease or condition is insulin-dependent diabetes mellitus (IDDM), the antigen may be glutamic acid decarboxylase (GAD), insulin, or IA-2 (a protein tyrosine phosphatase-like molecule).

Preferably, if the disease or condition is Goodpasture's syndrome or renal vasculitis, the antigen may be renal antigen or endothelial antigen.

Preferably, if the disease or condition is myasthenia gravis, the antigen may be the acetyl choline receptor

Preferably, if the disease or condition is sympathetic ophthalmia, the antigen may be ocular antigen.

Preferably, if the disease or condition is a myelin wasting disease, such as MS, the antigen may be myelin, MBP (myelin basic protein), PLP (proteolipid protein), or MOG (myelin oligodendrocyte glycoprotein).

Preferably, if the disease or condition is autoimmune haemolytic anaemia, the antigen may be red cell antigen.

Preferably, if the disease or condition is idiopathic leucopenia, the antigen may be leukocyte antigen.

Preferably, if the disease or condition is ulcerative colitis, the antigen may be a food antigen or a viral antigen.

Preferably, if the disease or condition is dermatomyositis, the antigen may be smooth muscle antigen.

Preferably, if the disease or condition is scleroderma, the antigen may be connective tissue antigen.

Preferably, if the disease or condition is mixed connective tissue disease, the antigen may be connective tissue antigen.

Preferably, if the disease or condition is irritable bowel syndrome, the antigen may be a food antigen.

Preferably, if the disease or condition is SLE, the antigen may be histone proteins or immunoglobulin heavy chain.

Preferably, if the disease or condition is Hashimoto's disease, primary myxoedema or thyrotoxicosis the antigen may be thyroid antigen.

Preferably, if the disease or condition is thyroid autoimmune disease or thyroiditis, the antigen may be a thyroid hormone such as thyroglobulin.

Preferably, if the disease or condition is Behcet's disease, the antigen may be Sag (S antigen from the eye), HLA-B44, B51, or HSP65.

Preferably, if the disease or condition is Coeliac disease/Dermatitis herpetiformis, the antigen may be gliadin. Rather than use whole gliadin, it may be useful to use a fraction of gliadin which is able to down regulate gliadin-specific T-cell proliferation. A suitable fraction may be the a fraction disclosed in Maurano et al (2001) Scand. J. Immunol. 53, 290-295, incorporated herein by reference.

Preferably, if the disease or condition is rheumatoid arthritis, the antigen may be type II collagen or an HSP (heat shock protein).

Preferably, if the disease or condition is demyelinating disease, the antigen may be myelin.

The methods of the invention can be used to retolerise a patient to an antigen. For example, in an autoimmune disease or condition which is a result of a viral infection, the antigen may be a self-HSP that is similar to a viral HSP.

The treatment is believed to combat the undesirable autoimmune response directly, as well as treating the symptoms by directing T cells away from a pro-inflammatory role.

Without being bound by theory, the inventor believes that the methods of the present invention may affect the programming of T cells so that they become regulatory or suppressive T cells rather than pro-inflammatory T cells. When a T cell that has been modulated by an antigen presenting cell in the presence of an agent which raises the effective cAMP concentration in a monocyte cell, such as a prostaglandin, and GMCSF and an antigen further encounters that same antigen, the T cell will release a suppressive cytokine such as IL-10. Treatment with an agent which raises the effective cAMP concentration in a monocyte cell, such as a prostaglandin, and GMCSF is thus believed to prevent or minimise an inflammatory response to that antigen from developing. Thus treatment with the said agent, such as a prostaglandin, and GMCSF, and optionally an antigen, eg a self-antigen, or a derivative thereof, can be used prophylactically, or as soon as the first symptoms of, eg an autoimmune disease, appear.

Furthermore, it will be appreciated that because T cells are present throughout the body they may be programmed or primed at a site remote from their ultimate site of action. Accordingly, in one embodiment of the invention, any one or all of the agent which raises the effective cAMP concentration in a monocyte cell, GMCSF or derivative thereof, and antigen or derivative thereof may be administered at a site distant from the site of disease.

Similarly, unlike other forms of treatment of certain autoimmune diseases, the method may be helpful in preventing inflammatory responses before they start. Thus, the method may be useful in treating patients who, for example because of their age or genetic factors, are predisposed to an autoimmune disease before any inflammatory symptoms show.

The invention also includes inducing tolerance to an antigen in a patient for inhibiting or dampening an immune or inflammatory response in the patient. By “inhibition or dampening” we include increasing the level of IL-10 which leads to an increase in the Th2 response, a decrease in the Th1 response, or an increase in T regulatory cells.

Whether or not a particular patient is one who is expected to benefit from treatment may be determined by the physician.

An effect of the treatment of a patient with an agent which raises the effective cAMP concentration in a monocyte cell, such as a prostaglandin or agonist thereof, and GMCSF or a derivative thereof may be the facilitation or improvement of tolerance to an antigen. The antigen may be one which is foreign to the patient or a self-antigen, and may be an antigen that is administered to the patient.

It is appreciated that the induction of tolerance to an antigen in a patient upon administration of the said agent, such as a prostaglandin or an agonist thereof, and GMCSF or a derivative thereof may lead to non-specific immune suppression. Thus, the invention includes a method of inducing tolerance to an antigen in a patient to create a state of immune suppression in the patient, the method comprising administering to the patient an agent which raises the effective cAMP concentration in a monocyte cell, such as a prostaglandin or agonist thereof, and GMCSF or a derivative thereof. Such a state of immune suppression is characterised by raising the threshold of a cell-mediated immune response to any antigenic stimulus.

Thus, it will be seen that the invention provides the use of the combination of an agent which raises the effective cAMP concentration in a monocyte cell, such as a prostaglandin or agonist thereof, and GMCSF or a derivative thereof as an immunosuppressant.

It is also appreciated that the induction of tolerance to an antigen in a patient upon administration of the said agent, such as a prostaglandin or agonist thereof, GMCSF or derivative thereof, and the antigen or a derivative thereof, may lead to antigen-specific immune suppression. Thus, the invention includes a method of inducing tolerance to an antigen in a patient to create a state of antigen-specific immune suppression in the patient, the method comprising administering to the patient the said agent, such as a prostaglandin or agonist thereof, GMCSF or derivative thereof, and the specific antigen or a derivative thereof. Such a state of antigen-specific immune suppression is characterised by raising the threshold of a cell-mediated immune response to stimulus by the specific antigen.

The invention therefore also provides the use of the combination of an agent which raises the effective cAMP concentration in a monocyte cell, such as a prostaglandin or agonist thereof, GMCSF or derivative thereof, and an antigen or a derivative thereof, as an immunosuppressant for that antigen.

The said agent, the GMCSF or derivative thereof, and the antigen or derivative thereof may be administered in any order. Preferably, they are co-administered. However, they may be administered so that the GMCSF or derivative thereof can take effect in the accessory cells prior to administration of the said agent, such as a prostaglandin or agonist thereof, and antigen. For example, the said agent, such as a prostaglandin or agonist thereof, and the antigen or derivative may be administered substantially simultaneously, for example in the same composition, with the GMCSF administered separately. The GMCSF may be administered before, after or substantially simultaneously with the said agent, such as a prostaglandin or agonist thereof, and the antigen or derivative. The order and timing of administration may be determined by the physician using knowledge of the properties of the antigen, the agent which raises the effective cAMP concentration in a monocyte cell and GMCSF. For example, the prostaglandin (such as misoprostol) may be active over a period of 4 hours following administration. Thus, suitable timings of administration can readily be devised from this information.

Where the tolerance to an antigen is desired to be localised to a particular organ, for example to the skin or the bronchial tree and lungs, it is preferred if the said agent, such as a prostaglandin or agonist thereof, and/or the GMCSF or derivative thereof and/or the antigen or derivative thereof is administered locally at the site of the condition. The said agent may be administered as a gel or cream or vapour or spray or in a “patch” in the case of a condition localised to the skin, or as an inhaled vapour or spray where the site is the lungs or bronchial tree.

The said agent and/or the GMCSF or derivative thereof and/or the antigen or derivative thereof may be administered systemically. For example, antigens presented locally to the mucosal immune system, eg via a suppository, are expected to act at mucosal sites remote from the site of administration.

The invention includes the administration of an agent which raises the effective cAMP concentration in a monocyte cell and/or GMCSF or derivative thereof and/or an antigen or derivative thereof to a mucosal site remote from the site of inflammation eg they could be co-administered as a suppository in the case of arthritis. This embodiment may be particularly advantageous as pathologic changes in the gastrointestinal tract may be associated with clinical complaints in multiple organs, including the musculoskeletal system (Alghafeer & Sigal, Bulletin on the Rheumatic Diseases, 51(2): http://www.arthritis.org/research/bulletin/vol51no2/51_{—}2_printable.asp, incorporated herein by reference). Some reactive arthritis can be triggered by inflammatory bowel diseases, and lymphocytes from the gut mucosa have been reported to migrate to joint tissue in enteropathic arthritis (Salmi & Jalkanen (2001) J Immunol., 166(7): 4650-7, incorporated herein by reference).

Thus, it is appreciated that the antigen or derivative thereof may be administered to a patient by a variety of means. For example, it can be administered via a mucosal surface of the patient, such as the rectal mucosal surfaces, eg as a suppository; it may be administered via the vagina eg in a pessary; it may be administered via the skin, eg as a gel of cream or patch; it may be administered to the lungs, eg as an aerosol (typically for lung disorders); or orally, eg as a tablet or capsule, (usually for delayed release in the gut).

Without being bound by theory, it is believed that the administered antigen or derivative thereof is transported to lymphoid tissues such as the lymph nodes in the lymph system or Peyer's patches in the submucosa of the small intestine. Thus, any form of delivery to these tissues is contemplated. The antigen or derivative thereof is presented to circulating T cells by an APC in a tolerising environment of raised IL-10 created by the prostaglandin and the GMCSF. Furthermore, administration of the prostaglandin and GMCSF increases the likelihood that the circulating T cells are regulatory T cells.

The GMCSF or derivative thereof may be administered by any suitable route. The GMCSF or derivative thereof may reach the desired site of action, which is typically the monocytes in relation to the present invention, using many different routes of administration. Typically, in one embodiment, the GMCSF or derivative thereof is administered systemically. Suitable forms of systemic administration include oral, transcutaneous, subcutaneous or intravenous administration, or by suppository.

It may be convenient to administer the GMCSF or derivative thereof locally. Thus, the GMCSF may be delivered locally, such as on the skin, using, for example, a gel or cream or vapour or spray or in a “patch” as described above in relation to the administration of the prostaglandin or agonist thereof. Similarly, in the case of administration to the bronchial tree or lungs it may be administered as a spray or vapour.

In preferred embodiments of the invention, the agent which raises the effective cAMP concentration in a monocyte cell, such as a prostaglandin or agonist thereof, and/or the GMCSF or derivative thereof and/or the antigen or a derivative thereof may be combined in the same formulation for delivery simultaneously. Thus, the agent which raises the effective cAMP concentration in a monocyte cell, such as a prostaglandin or agonist thereof, and/or the GMCSF or derivative thereof and/or the antigen may be combined in a gel or a cream or a vapour or spray or “patch” or suppository and administered together to the patient.

Preferably, a suppository has an enteric coating which only releases the active agents in the bowel when the pH has risen. This sort of preparation has been successful in the delivery of glucocorticoids to the bowel (data sheet for Entocort CR).

Alternatively, the agent which raises the effective cAMP concentration in a monocyte cell and/or the GMCSF or derivative thereof and/or the antigen or derivative thereof, may be administered in a capsule or other suitable form that is swallowed. The capsule or other suitable form has an enteric coating which is pH sensitive, leading to release at an appropriate point in the gastrointestinal tract where it is desired to do so, typically the distal ileum or colon.

Alternatively, the said agent, such as a prostaglandin or agonist thereof, and/or the GMCSF and/or the antigen or derivative thereof, may be administered directly to the colon or distal ileum using a non-soluble tube or pipe system, such as produced by Egalet.

It is appreciated that the said agent and/or the GMCSF and/or the antigen may be administered at the same or different sites, and by the same or different modes of administration.

In one embodiment the agent which raises the effective cAMP concentration in a monocyte cell, such as a prostaglandin or agonist thereof, is administered orally. In particular the prostaglandin or agonist thereof is a prostaglandin analogue which has been modified to reduce its catabolism and which is orally available (such as misoprostol).

Thus, in a preferred embodiment, the method of the invention makes use of the oral administration of a prostaglandin analogue which has been modified to reduce its catabolism and which is orally available (such as misoprostol), and the antigen or derivative thereof is also administered orally. The advantages of oral administration is that it generally has good compliance compared to other modes of administration.

The inventor believes that the combination of GMCSF or derivative thereof with the orally available prostaglandin or agonist thereof will mean that a lower dose of oral prostaglandin will be required than in the absence of GMCSF. Moreover, this combination of prostaglandin and GMCSF has been shown to give a prolonged effect even after its absence (see Examples 2 and 3) which is believed to be due to the presence of GMCSF which is a differentiating agent. It is believed by the inventor that this will have the advantage of reducing side effects caused by the oral prostaglandin or agonist thereof, such as muscle cramps.

Preferably, the combination of GMCSF and prostaglandin or agonist thereof, comprises GMCSF and PGE. Typically, 0.1-100 μg of PGE and 1-250 μg GMCSF in 5 ml saline would be administered.

Alternatively, the combination of GMCSF and prostaglandin or agonist thereof, may comprise GMCSF and a 19-hydroxy PGE. Typically, 0.1-100 μg of 19 hydroxy PGE and 1-250 μg GMCSF in 5 ml saline would be administered.

A suitable dose of sargramostim is 250 μg/m² daily.

Typically, 100 to 800 μg, more preferably 100 to 400 μg, of misoprostol is administered orally daily.

Typically, the antigen or derivative thereof is administered in a dose between about 100 ng and about 100 mg, more typically about 100 μg.

A third aspect of the invention provides a composition comprising an agent which raises the effective cAMP concentration in a monocyte cell and GMCSF or a derivative thereof for use in medicine. The invention also includes a composition comprising an agent which raises the effective cAMP concentration in a monocyte cell, GMCSF or a derivative thereof, and an antigen to which it is desired to induce tolerance in a patient or a derivative thereof, for use in medicine. The composition is therefore packaged and presented for use in medicine. The composition may be used in human or veterinary medicine; preferably, it is used in human medicine.

Preferably, the use according to the third aspect is in treating an aberrant or undesired immune or inflammatory response in the patient, as described above.

The preferences for the agent as said (eg a prostaglandin or agonist thereof), the GMCSF or derivative thereof, and the antigen or derivative thereof for the third aspect of the invention are the same as for the first aspect of the invention.

In an embodiment, the composition may further comprise a monocyte chemotactic agent, such as those described above.

In an additional or alternative embodiment, the composition may further comprise a PDE inhibitor, such as those described above.

A fourth aspect of the invention provides the use of an agent which raises the effective cAMP concentration in a monocyte cell in the manufacture of a medicament for inducing tolerance to an antigen in a patient wherein the patient is administered GMCSF or a derivative thereof. Thus, the patient may already have been administered the GMCSF or derivative thereof before administration of the said agent, or is administered the GMCSF or derivative thereof at the same time as the said agent or will be administered the GMCSF or derivative thereof after administration of the said agent.

A fifth aspect of the invention is the use of GMCSF or derivative thereof in the manufacture of a medicament for inducing tolerance to an antigen in a patient wherein the patient is administered an agent which raises the effective cAMP concentration in a monocyte cell. Thus, the patient may already have been administered the said agent thereof before administration of the GMCSF or derivative thereof, or is administered the said agent at the same time as the GMCSF or derivative thereof or will be administered the said agent after administration of the GMCSF or derivative thereof.

A sixth aspect of the invention provides the use of a combination of an agent which raises the effective cAMP concentration in a monocyte cell and GMCSF or a derivative thereof in the manufacture of a medicament for inducing tolerance to an antigen in a patient. Thus, the said agent and GMCSF or derivative thereof may be combined in the same medicament before administration to the patient.

A seventh aspect of the invention provides the use of an agent which raises the effective cAMP concentration in a monocyte cell in the manufacture of a medicament for inducing tolerance to an antigen in a patient wherein the patient is administered GMCSF or derivative thereof and the antigen or a derivative thereof. Thus, the patient may already have been administered the GMCSF or derivative thereof and the antigen or derivative thereof before administration of the said agent, or is administered the GMCSF or derivative thereof and the antigen or derivative thereof at the same time as the said agent, or will be administered the GMCSF or derivative thereof and the antigen or derivative thereof after administration of the said agent.

An eighth aspect of the invention is the use of GMCSF or derivative thereof in the manufacture of a medicament for inducing tolerance to an antigen in a patient wherein the patient is administered an agent which raises the effective cAMP concentration in a monocyte cell and the antigen or a derivative thereof. Thus, the patient may already have been administered the said agent and the antigen or derivative thereof before administration of the GMCSF or derivative thereof, or is administered the said agent and the antigen or derivative thereof at the same time as the GMCSF or derivative thereof, or will be administered the said agent and the antigen or derivative thereof after administration of the GMCSF or derivative thereof.

A ninth aspect of the invention is the use of an antigen or a derivative thereof in the manufacture of a medicament for inducing tolerance to the antigen in a patient wherein the patient is administered an agent which raises the effective cAMP concentration in a monocyte cell and GMCSF or derivative thereof. Thus, the patient may already have been administered the said agent and GMCSF or derivative thereof before administration of the antigen or derivative thereof, or is administered the said agent and the GMCSF or derivative thereof at the same time as the antigen or derivative thereof, or will be administered the said agent and the GMCSF or derivative thereof after administration of the antigen or derivative thereof.

A tenth aspect of the invention provides the use of an agent which raises the effective cAMP concentration in a monocyte cell and GMCSF or derivative thereof in the manufacture of a medicament for inducing tolerance to an antigen in a patient wherein the patient is administered the antigen or a derivative thereof. Thus, the patient may already have been administered the antigen or derivative thereof before administration of the said agent and GMCSF or derivative thereof, or is administered the antigen or derivative thereof at the same time as the said agent and GMCSF or derivative thereof, or will be administered the antigen or derivative thereof after administration of the said agent and GMCSF or derivative thereof.

An eleventh aspect of the invention is the use of a GMCSF or derivative thereof and an antigen or a derivative thereof in the manufacture of a medicament for inducing tolerance to the antigen in a patient wherein the patient is administered an agent which raises the effective cAMP concentration in a monocyte cell. Thus, the patient may already have been administered the said agent before administration of the GMCSF or derivative thereof and the antigen or derivative thereof, or is administered the said agent at the same time as the GMCSF or derivative thereof and the antigen or derivative thereof, or will be administered the said agent after administration of the GMCSF or derivative thereof and the antigen or derivative thereof.

A twelfth aspect of the invention provides the use of an agent which raises the effective cAMP concentration in a monocyte cell, and an antigen or a derivative thereof, in the manufacture of a medicament for inducing tolerance to the antigen in a patient wherein the patient is administered GMCSF or derivative thereof. Thus, the patient may already have been administered the GMCSF or derivative thereof before administration of the said agent and the antigen or derivative thereof, or is administered the GMCSF or derivative thereof at the same time as the said agent and the antigen or derivative thereof, or will be administered the GMCSF or derivative thereof after administration of the said agent and the antigen or derivative thereof.

A thirteenth aspect of the invention provides the use of a combination of an agent which raises the effective cAMP concentration in a monocyte cell, GMCSF or derivative thereof and an antigen or a derivative thereof in the manufacture of a medicament for inducing tolerance to the antigen in a patient. Thus, the said agent, GMCSF or derivative thereof and the antigen or a derivative thereof may be combined in the same medicament before administration to the patient.

Preferably, the use according to the fourth to the thirteenth aspects is in treating an aberrant or undesired immune or inflammatory response in the patient, as described above.

The preferences for the agent which raises the effective cAMP concentration in a monocyte cell, such as a prostaglandin or agonist thereof, the GMCSF or derivative thereof, the antigen or derivative thereof, routes of administration, doses and so on for the fourth to the thirteenth aspects of the invention are the same as for the first aspect of the invention.

It is appreciated that the medicaments described in the fourth to the thirteenth aspects of the invention may also comprise a monocyte chemotactic agent and/or a PDE inhibitor. It is further appreciated that the medicaments described in the fourth to the thirteenth aspects of the invention may be for inducing tolerance to an antigen in a patient who has been administered a monocyte chemotactic agent and/or a PDE inhibitor. The preferences for the monocyte chemotactic agent and the PDE inhibitor are the same as for the first aspect of the invention.

A fourteenth aspect of the invention provides a therapeutic system for inducing tolerance to an antigen, the system comprising an agent which raises the effective cAMP concentration in a monocyte cell and GMCSF or derivative thereof. The therapeutic system may also be termed a “kit of parts”.

The invention includes a therapeutic system for inducing tolerance to an antigen, the system comprising an agent which raises the effective cAMP concentration in a monocyte cell, GMCSF or a derivative thereof, and the antigen to which it is desired to induce tolerance, or a derivative thereof.

Preferably, the therapeutic system contains a preferred agent, such as a prostaglandin or agonist thereof, as defined in the first aspect of the invention. Still preferably, the therapeutic system contains a preferred GMCSF or derivative thereof as defined in the first aspect of the invention. If the therapeutic system contains an antigen or derivative thereof it is preferably as defined in the first aspect of the invention.

The therapeutic system or kit of parts may suitably contain the agent which raises the effective cAMP concentration in a monocyte cell, the GMCSF or derivative thereof, and the antigen or derivative thereof packaged and presented in suitable formulations for use in combination, either for administration simultaneously or for administration which is separated in time. Thus, for example, in one embodiment where the said agent, such as a prostaglandin or agonist thereof, GMCSF or derivative thereof, and antigen or derivative thereof are for simultaneous administration locally to the skin, the therapeutic system may contain a gel or cream or spray or vapour or “patch” which contains a combination of the said agent, such as a prostaglandin or agonist thereof, GMCSF or derivative thereof and the antigen or derivative thereof. Alternatively, in another embodiment where the said agent, such as a prostaglandin or agonist thereof, GMCSF or derivative thereof and antigen or derivative thereof are for separate administration in a particular treatment regime, they are packaged or formulated separately. For example, the said agent, such as a prostaglandin or agonist thereof, may be formulated for administration locally using a cream or gel or spray or vapour or “patch”, and GMCSF or derivative thereof and the antigen or derivative thereof are packaged or formulated for intravenous or oral administration.

It is appreciated that the therapeutic system may also comprise a suitably packaged and presented monocyte chemotactic agent and/or a PDE inhibitor, such as those described above with respect to the first aspect of the invention.

The formulations of the agent which raises the effective cAMP concentration in a monocyte cell, such as prostaglandin or agonist thereof, alone or GMCSF or derivative thereof alone or the antigen or derivative thereof alone, or any combination of any two of them, or all three of them, may conveniently be presented in unit dosage form and may be prepared by any of the methods well known in the art of pharmacy. Such methods include the step of bringing into association the active ingredients used in the invention with the carrier which constitutes one or more accessory ingredients. In general the formulations are prepared by uniformly and intimately bringing into association the active ingredient with liquid carriers or finely divided solid carriers or both, and then, if necessary, shaping the product.

Formulations in accordance with the present invention suitable for oral administration (eg of the GMCSF or of a suitable agent which raises the effective cAMP concentration in a monocyte cell, such as a prostaglandin or agonist thereof, or antigen) may be presented as discrete units such as capsules, cachets or tablets, each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

A tablet may be made by compression or molding, optionally with one or more accessory ingredients. Compressed tablets may be prepared by compressing in a suitable machine the active ingredient in a free-flowing form such as a powder or granules, optionally mixed with a binder (eg povidone, gelatin, hydroxypropylmethyl cellulose), lubricant, inert diluent, preservative, disintegrant (eg sodium starch glycolate, cross-linked povidone, cross-linked sodium carboxymethyl cellulose), surface-active or dispersing agent. Molded tablets may be made by molding in a suitable machine a mixture of the powdered compound moistened with an inert liquid diluent. The tablets may optionally be coated or scored and may be formulated so as to provide slow or controlled release of the active ingredient therein using, for example, hydroxypropylmethylcellulose in varying proportions to provide desired release profile.

Preferred unit dosage formulations are those containing a daily dose or unit, daily sub-dose or an appropriate fraction thereof, of an active ingredient.

It should be understood that in addition to the ingredients particularly mentioned above the formulations of this invention may include other agents conventional in the art having regard to the type of formulation in question, for example those suitable for oral administration may include flavouring agents.

For local administration to the skin, it may be convenient to formulate the said agent, such as a prostaglandin or agonist thereof, and/or GMCSF or derivative thereof and/or antigen or derivative thereof in combination with a dispersion agent or an agent which allows for increased transdermal or transmucosal transfer or penetration, such a dimethyl sulphoxide (DMSO) and the like. Suitable agents are ones which are compatible with the said agent, such as a prostaglandin or agonist thereof and/or GMCSF or derivative thereof (eg are solvents thereof).

The patient on which the method or medicament is used is preferably a human although the patient may be any mammal such as a cat, dog, horse, cow, sheep, horse, pig and so on.

It will be appreciated that the method or medicament may be used before symptoms indicating a need to induce tolerance of an antigen becomes apparent in the patient to be treated, or, either alternatively or in addition, the using of the method or medicament may be used after symptoms or signs become apparent. Thus, in the case of a patient receiving an organ or tissue transplant, it may be beneficial to administer the agent which raises the effective cAMP concentration in a monocyte cell, such as a prostaglandin or agonist thereof, GMCSF or derivative thereof, and optionally the antigen or derivative thereof, before the transplantation surgery is started. It may be further beneficial to continue the administration during or after completion of the transplant or graft surgery. The necessary dosage may be determined by the physician, according to the degree of tolerance that is required.

It will further be appreciated that each of the agent which raises the effective cAMP concentration in a monocyte cell, such as a prostaglandin or agonist thereof, the GMCSF or derivative thereof, and optionally the antigen or derivative thereof, may be administered as a single dose, or in multiple smaller doses which achieve the same therapeutic effect. The frequency of administration may vary according to the convenience of the physician administering the dose or the patient.

It is appreciated that it may be preferable to minimise the exposure of the patient to other antigens other than the one to which it is desired to induce tolerance. In some cases, this may include keeping the patient in an isolation “bubble” as is known in the art for immunosuppressed patients.

It is appreciated that tolerance to more than one antigen may be desired. Therefore reference to methods, uses and compositions comprising an antigen to which it is desired to induce tolerance, may include two or three or four or five or more antigens to which it is desired to induce tolerance.

Pregnancy is likely to be a contraindication for the present invention. In fact, pregnancy is a contraindication for several prostaglandins including misoprostol. Cytotec (misoprostol) does not cause hypotension, but this may be a possible risk with the method of the invention.

The inventor has shown that prostaglandin E and GMCSF cause enhanced expression of IL-10, granulysin, COX-2, CD86 and CD14 in cells of the macrophage/monocyte lineage. By “cells of the macrophage/monocyte lineage” we include cells that are derived from monocyte precursors and include macrophages, monocytes and dendritic cells.

The present invention also includes a method of stimulating or enhancing IL-10 expression in, and secretion from, cells of the macrophage/monocyte lineage comprising administering an agent which raises the effective cAMP concentration in a monocyte cell, such as a prostaglandin of agonist thereof, and GMCSF or a derivative thereof. The stimulation can be in vitro, or in vivo.

The method of stimulating or enhancing IL-10 expression in, and secretion from, cells of the macrophage/monocyte lineage may further comprise administering a monocyte chemotactic agent and/or a PDE inhibitor.

The stimulation or enhancement of IL-10 secretion in vivo may be beneficial in conditions such as transplants, autoimmune diseases and allergies as described previously with respect to the first aspect of the invention.

The present invention also provides the use of an agent which raises the effective cAMP concentration in a monocyte cell, such as a prostaglandin or agonist thereof, in the manufacture of a medicament for stimulating or enhancing IL-10 expression in, and secretion from, cells of the macrophage/monocyte lineage in a patient wherein the patient is administered GMCSF or a derivative thereof. Thus, the patient may already have been administered the GMCSF before administration of the said agent, or is administered the GMCSF at the same time as the said agent thereof or will be administered the GMCSF after administration of the prostaglandin or agonist thereof.

The present invention further provides the use of GMCSF or a derivative thereof in the manufacture of a medicament for stimulating or enhancing IL-10 expression in, and secretion from, cells of the macrophage/monocyte lineage in a patient wherein the patient is administered an agent which raises the effective cAMP concentration in a monocyte cell, such as a prostaglandin or agonist thereof. Thus, the patient may already have been administered the said agent before administration of the GMCSF, or is administered the said agent at the same time as the GMCSF or will be administered the said agent after administration of the GMCSF.

The present invention additionally provides the use of a combination of an 15 agent which raises the effective cAMP concentration in a monocyte cell, such as a prostaglandin or agonist thereof, and GMCSF or a derivative thereof in the manufacture of a medicament for stimulating or enhancing IL-10 expression in, and secretion from, cells of the macrophage/monocyte lineage in a patient. Thus, the said agent, such as a prostaglandin or agonist thereof and GMCSF may be combined in the same medicament before administration to the patient.

The present invention also includes a method of stimulating or enhancing granulysin expression in, and secretion from, cells of the macrophage/monocyte lineage comprising administering an agent which raises the effective cAMP concentration in a monocyte cell, such as a prostaglandin or an agonist thereof, and GMCSF or a derivative thereof. The stimulation can be in vitro or in vivo, for example in a patient in need of the increased antiviral activity of granulysin.

The method of stimulating or enhancing granulysin expression in, and secretion from, cells of the macrophage/monocyte lineage may further comprise administering a monocyte chemotactic agent and/or a PDE inhibitor.

The stimulation or enhancement of granulysin secretion in vivo may be beneficial in conditions such as transplants, autoimmune diseases and allergies as described previously with respect to the first aspect of the invention.

The present invention also provides the use of an agent which raises the effective cAMP concentration in a monocyte cell, such as a prostaglandin or agonist thereof, in the manufacture of a medicament for stimulating or enhancing granulysin expression in, and secretion from, cells of the macrophage/monocyte lineage in a patient wherein the patient is administered GMCSF or a derivative thereof. Thus, the patient may already have been administered the GMCSF before administration of the said agent, or is administered the GMCSF at the same time as the said agent or will be administered the GMCSF after administration of the said agent.

The present invention further provides the use of GMCSF or a derivative thereof in the manufacture of a medicament for stimulating or enhancing granulysin expression in, and secretion from, cells of the macrophage/monocyte lineage in a patient wherein the patient is administered an agent which raises the effective cAMP concentration in a monocyte cell, such as a prostaglandin or agonist thereof. Thus, the patient may already have been administered the said agent before administration of the GMCSF, or is administered the said agent at the same time as the GMCSF or will be administered the said agent after administration of the GMCSF.

The present invention additionally provides the use of a combination of an agent which raises the effective cAMP concentration in a monocyte cell, such as a prostaglandin or agonist thereof, and GMCSF or a derivative thereof in the manufacture of a medicament for stimulating or enhancing granulysin expression in, and secretion from, cells of the macrophage/monocyte lineage in a patient. Thus, the said agent and GMCSF may be combined in the same medicament before administration to the patient.

The stimulation or enhancement of granulysin secretion in vivo may further be beneficial as an anti-viral treatment against, for example, herpes simplex virus or human papilloma virus. Thus the combination of an agent which raises the effective cAMP concentration in a monocyte cell, such as a prostaglandin or an agonist thereof, and GMCSF or a derivative thereof, optionally with a monocyte chemotactic agent such as MCP-1 or MIP-1α, can be applied topically to the skin to prevent the development of, or to treat, cold sores or warts.

Thus the invention includes a method of treating a viral infection in a patient comprising administering to the patient an agent which raises the effective cAMP concentration in a monocyte cell, such as a prostaglandin or an agonist thereof, and GMCSF or a derivative thereof. The method may further include administering a monocyte chemotactic agent and/or a PDE inhibitor to the patient.

The invention also includes a method of treating cold sores or warts comprising administering to the cold sores or warts an agent which raises the effective cAMP concentration in a monocyte cell, such as a prostaglandin or an agonist thereof, and GMCSF or a derivative thereof. The method may further include administering a monocyte chemotactic agent and/or a PDE inhibitor to the cold sores or warts.

The present invention also provides the use of an agent which raises the effective cAMP concentration in a monocyte cell, such as a prostaglandin or agonist thereof, in the manufacture of a medicament for treating a viral infection in a patient, wherein the patient is administered GMCSF or a derivative thereof. Thus, the patient may already have been administered the GMCSF before administration of the said agent, or is administered the GMCSF at the same time as the said agent or will be administered the GMCSF after administration of the said agent.

The present invention further provides the use of GMCSF or a derivative thereof in the manufacture of a medicament for treating a viral infection in a patient, wherein the patient is administered an agent which raises the effective cAMP concentration in a monocyte cell, such as a prostaglandin or agonist thereof. Thus, the patient may already have been administered the said agent before administration of the GMCSF, or is administered the said agent at the same time as the GMCSF or will be administered the said agent thereof after administration of the GMCSF.

The present invention additionally provides the use of a combination of an agent which raises the effective cAMP concentration in a monocyte cell, such as a prostaglandin or agonist thereof, and GMCSF or a derivative thereof in the manufacture of a medicament for treating a viral infection in a patient. Thus, the said agent and GMCSF may be combined in the same medicament before administration to the patient.

The stimulation or enhancement of granulysin secretion in vivo may also be beneficial as an anti-tumour treatment, not least because of granulysin's antiviral activity. For example, the stimulation or enhancement of granulysin secretion may be beneficial for treating early stage skin cancer or cervical cancer.

The present invention also includes a method of stimulating or enhancing COX-2 expression in cells of the macrophage/monocyte lineage comprising administering an agent which raises the effective cAMP concentration in a monocyte cell, such as a prostaglandin or agonist thereof, and GMCSF or a derivative thereof. The stimulation can be in vitro, or in vivo.

The stimulation or enhancement of COX-2 expression in vivo may be beneficial in conditions such as transplants, autoimmune diseases and allergies as described previously with respect to the first aspect of the invention, as it is believed to be involved in maintaining the tolerising and antimicrobial phenotype induced by the prostaglandin and the GMCSF.

The present invention also provides the use of an agent which raises the effective cAMP concentration in a monocyte cell, such as a prostaglandin or agonist thereof, in the manufacture of a medicament for stimulating or enhancing COX-2 expression in cells of the macrophage/monocyte lineage in a patient wherein the patient is administered GMCSF or a derivative thereof. Thus, the patient may already have been administered the GMCSF before administration of the said agent, or is administered the GMCSF at the same time as the said agent or will be administered the GMCSF after administration of the said agent.

The present invention further provides the use of GMCSF or a derivative thereof in the manufacture of a medicament for stimulating or enhancing COX-2 expression in cells of the macrophage/monocyte lineage in a patient wherein the patient is administered an agent which raises the effective cAMP concentration in a monocyte cell, such as a prostaglandin or agonist thereof. Thus, the patient may already have been administered the said agent before administration of the GMCSF, or is administered the said agent at the same time as the GMCSF or will be administered the said agent after administration of the GMCSF.

The present invention additionally provides the use of a combination of an agent which raises the effective cAMP concentration in a monocyte cell, such as a prostaglandin or agonist thereof and GMCSF or a derivative thereof in the manufacture of a medicament for stimulating or enhancing COX-2 expression in cells of the macrophage/monocyte lineage in a patient. Thus, the said agent and GMCSF may be combined in the same medicament before administration to the patient.

It will be appreciated that in all aspects and embodiments of the invention it is preferred that the agent which raises the effective cAMP concentration in a monocyte cell is a prostaglandin or agonist thereof.

All of the documents referred to herein are incorporated herein, in their entirety, by reference.

The invention will now be described in more detail with the aid of the following Figures and Examples.

FIG. 1

cDNA and amino acid sequence (FIGS. 1A and 1B, respectively) of human GMCSF, taken from Genbank Accession No. NM_{—}000758.

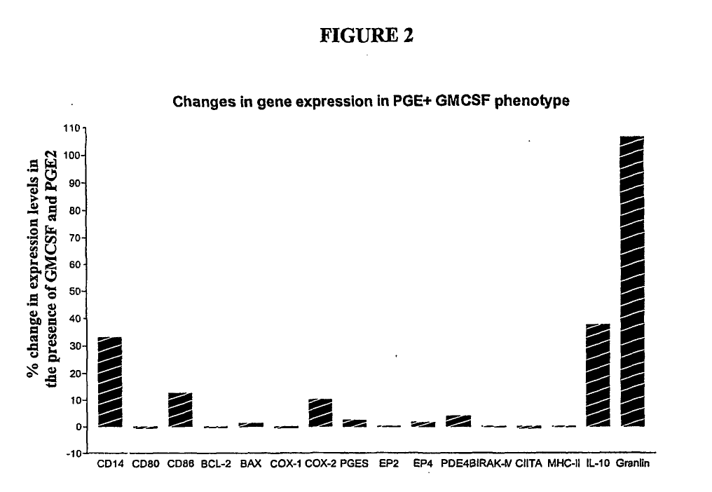

FIG. 2

FIG. 2 is a graph showing the effect of PGE and GMCSF on gene expression in U937 cells. Cells were treated for 4 hours with PGE2, with and without GMCSF, washed to remove the treatment, and incubated for a further 20 hours before the cells were pelleted and RNA extracted. The mRNA levels of CD14, CD80, CD86, BCL-2, BAX, COX-1 (cyclo-oygenase 1), COX-2, PGES (prostaglandin synthase), EP2 (a prostaglandin receptor), EP4 (a prostaglandin receptor), PDE4B (a phosphodiesterase), IRAK-UV, CIITA (MIC class II transactivator), MHC-II, IL-10 and granulysin (abbreviated to granlin), were measured. The graph indicates the percentage change in expression levels in the presence of GMCSF and PGE2.

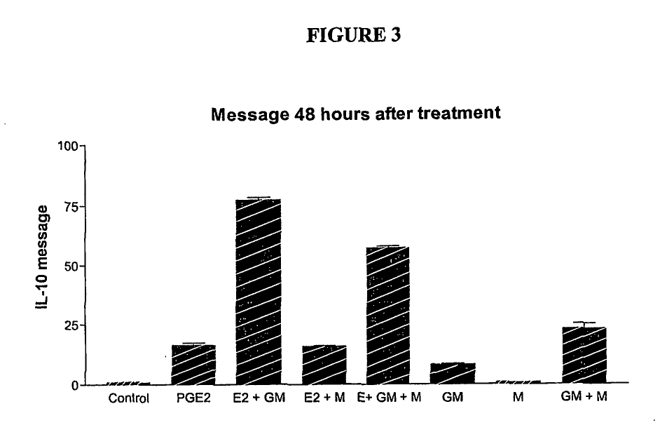

FIG. 3

FIG. 3 is a graph showing the synergistic effect of PGE and GMCSF on the production of IL-10 mRNA in U937 cells, and that this phenotype is maintained 48 hours after removal of the treatment. Cells were treated for 4 hours with the agents indicated below the graph, washed to remove the treatment, and incubated for a further 48 hours before the cells were pelleted and RNA extracted. PGE2, E2 and E all refer to prostaglandin E2; GM refers to GMCSF; and M refers to MCSF.

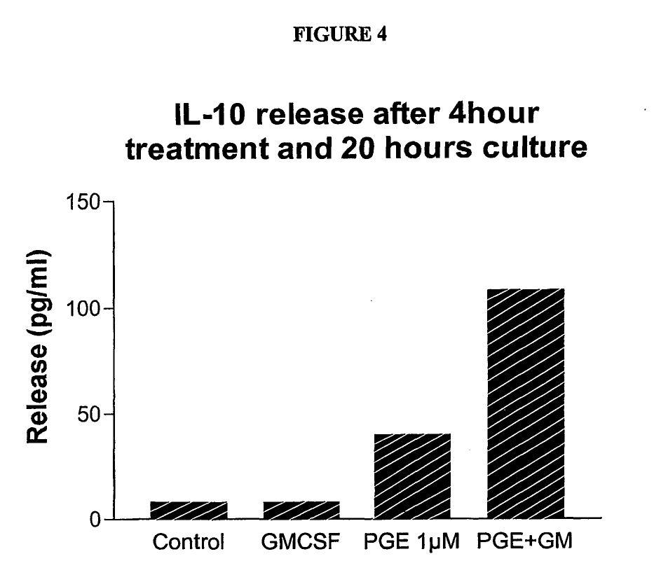

FIG. 4

FIG. 4 is a graph showing the synergistic effect of PGE and GMCSF on the release of IL-10 protein in U937 cells, and that this phenotype is maintained after removal of the treatment. Cells were treated for 4 hours with the agents indicated below the graph, washed to remove the treatment, and incubated for a further 20 hours before the medium was assayed for IL-10. PGE refers to prostaglandin E2, and GM refers to GMCSF.

FIG. 5

FIG. 5 is a diagram showing agents which control intracellular cAMP. Open arrows are effectively lowering intracellular cAMP levels. Solid arrow is stimulation. Combinations will be synergistic.

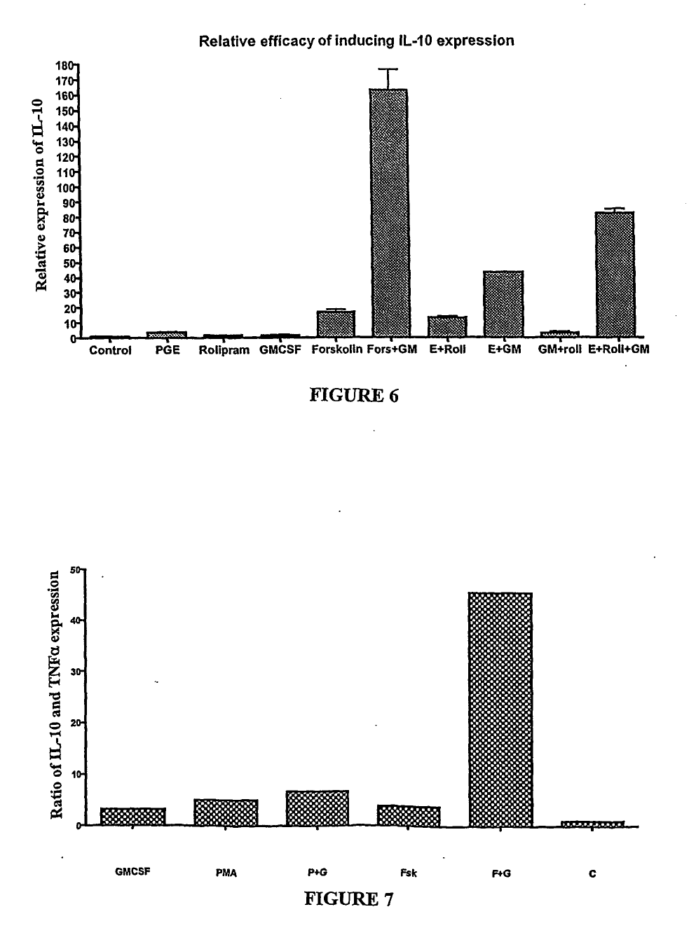

FIG. 6

FIG. 6 shows the relative efficacy of various agents in inducing IL-10 expression. See Example 4 for details.

FIG. 7

FIG. 7 shows the relative efficacy of various agents in inducing IL-10, expressed as a ratio of IL-10/TNFα mRNA expression. See Example 5 for details.

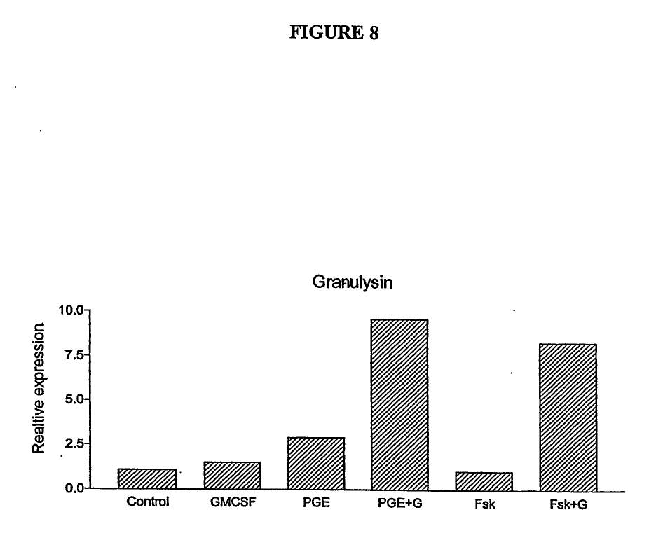

FIG. 8

FIG. 8 shows the relative efficacy of various agents and combinations of agents in inducing granulysin mRNA expression. See Example 6 for details.

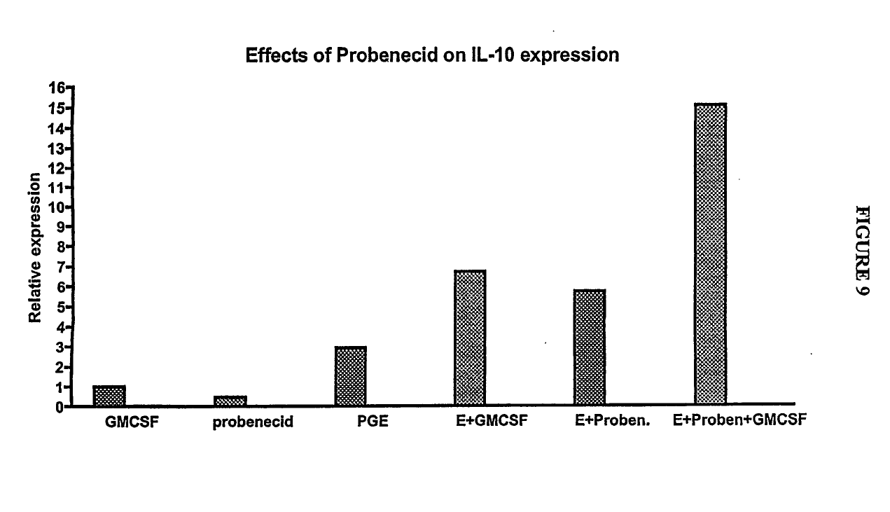

FIG. 9

FIG. 9 shows that there is a synergistic effect between a prostaglandin (PGE2) and GMCSF and probenicid on the expression of IL-10.

### EXAMPLE 1

### Prostaglandin E/GMCSF Synergism for Inducing Immunological Tolerance

There is growing evidence that prostaglandins of the E series are involved in immunological tolerance. This derives from their role in oral tolerance (the ability of the immune system to distinguish pathogenic and comensal organisms), their ability to modulate cytokine ratios, and their huge concentrations in human seminal plasma where tolerance for the spermatozoon is essential.

Prostaglandins are produced at most mucosal surfaces of the body that have to accommodate beneficial or harmless bacteria and yet mount a response to pathogens. Newberry et al (1999) have shown that 3A9 TCRα −/− mice expressing a T cell receptor that specifically recognises egg-white lysosyme do not mount an inflammatory response to this antigen unless prostaglandin synthesis is inhibited, in that case by inhibiting the inducible cyclooxygenase isoform COX-2. With the source of prostaglandin removed, and with exposure to the specific antigen, these mice develop a pathology resembling inflammatory bowel disease (Newberry et al., 1999). These experiments confirm earlier studies showing that non-steroidal anti-inflammatory drugs such as indomethacin, which have a primary effect of inhibiting prostaglandin synthesis, break tolerance (Scheuer et al, 1987; Louis et al., 1996).

Monocytes of the normal lamina propria have a distinct phenotype since they express CD86 but not CD80. When an inflammatory condition persists (e.g inflammatory bowel disease) the monocytes express CD80 (Rugtveit et al., 1997). The resident macrophages (CD80−ve CD86+ve) are thus distinguished from the recently recruited macrophages which are CD80+ve, CD86+ve.

Monocytes are major sources of many immunological mediators, including prostaglandins and as such will alter the cytokine environment for antigen presentation. PGE has a major effect on cytokines relevant to tolerance, stimulating the tolerogenic cytokine IL-10 (Strassmann et al., 1994) and inhibiting IL-12 (Kraan et al., 1995) which breaks tolerance. PGE will also have direct effects on the maturation of antigen-presenting dendritic cells, stimulating the production of cells that secrete increased IL-10 and diminished IL-12 (Kalinski et al., 1997).

A further indication of the importance of prostaglandins in ensuring essential tolerance is the very high (approximately millimolar) concentrations of both PGE and 19-hydroxy PGE in human seminal plasma. Clearly, immunological tolerance for spermatozoa entering the immunologically competent, and possibly infected, female genital tract is essential for the continuation of the species and levels of prostaglandin are such that many sub-epithelial, and even lymph-node cells will be affected. In this way, evolution has ensured immunological protection for the spermatozoa.

Previous experiments (Strassmann et al., 1994; Kraan et al., 1995) have required lipopolysaccharide (LPS) to be present for PGE to stimulate IL-10 production and in addition, the message for IL-10 was delayed by approximately 12 hours, both of these factors has been puzzling. The observations of the present invention suggest that LPS may have been stimulating the expression of GMCSF, which may account for both the delay and the subsequent IL-10-expression.

We now show that the major prostaglandin effects on tolerance inducing monocytes may be mediated by a synergism between a prostaglandin and GMCSF. The result of short term exposure to this combination results in a phenotype expressing greatly increased IL-10 but reduced levels of participants in antigen presentation such as CIITA and MHCII. Moreover, this change in phenotype is accompanied by enhanced expression of granulysin. This molecule has anti-microbial properties (Krensky 2000) and is normally thought of as a product of activated T cells—mediating antiviral activity that lyses infected cells (Hata et al. 2001; Ochoa et al. 2001; Smyth et al. 2001). Such an increase in innate defence molecules may compensate for the compromise of the adaptive immune system that necessarily accompanies tolerance induction. The phenotype is further characterised by a neutral effect on CD80 but a stimulation of CD86.

Experimental Details

U937 (human monocyte cell line) cells were grown in RPMI (PAA Laboratories) medium with 10% fetal calf serum added (PAA Laboratories). Cells were treated with prostaglandin E2 at 10⁻⁶ Molar with or without GMCSF with at 5 ng/ml for 4 hours. The treatment was removed and cells were cultured for a further 20 hours. Cells were pelleted and the mRNA was extracted with Tri reagent (Sigma, Poole, UK). Total RNA was obtained by addition of chloroform and subsequent isopropanol precipitation. RNA was reverse transcribed with reverse transcriptase (Applied Biosystems) and random hexamers (Applied Biosystems). Probes and primers for amplification and detection of IL-10 and a number of other molecules were designed using Primer Express (Applied Biosystems) and are as follows:

IL-10 primers
CTACGGCGCTGTCATCGAT
TGGAGCTTATTAAAGGCATTCTTCA
IL-10 probe
CTTCCCTGTGAAAACAAGAGCAAGGCC
BAX primers
CATGGAGCTGCAGAGGATGA
CTGCCACTCGGAAAAAGACCT
Bax Probe
TGCCGCCGTGGACACAGACTCC
BCL2 primers
CCGGGAGGCGACCGTAGT
GGGCTGCGCACCCTTTC
BCL2 probe
CGCCGCGCAGGACCAGGA
CD80 primers
TCCACGTGACCAAGGAAGTG
CCAGCTCTTCAACAGAAACATTGT
CD8O Probe
AAGAAGTGGCAACGCTGTCCTGTGG
CD86 primers
CAGACCTGCCATGCCAATT
TTCCTGGTCCTGCCAAAATACTA
CD86 Probe
CAAACTCTCAAAACCAAAGCCTGAGTGAGC
COX-1 primers
TGTTCGGTGTCCAGTTCCAATA
ACCTTGAAGGAGTCAGGCATGAG
COX-1 Probe
CGCAACCGCATTGCCATGGAGT
COX-2 primers
GTGTTGACATCCAGATCACATTTGA
GAGAAGGCTTCCCAGCTTTTGTA
COX-2 Probe
TGACAGTCCACCAACTTACAATGCTGACTATGG
EP2 primers
GAC CGC TTA CCT GCA GCT GTA C
TGA AGT TGC AGG CGA GCA
EP2 Probe
CCA CCC TGC TGC TGC TTC TCA TTG TCT
EP4 primers
ACGCCGCCTACTCCTACATG
AGAGGACGGTGGCGAGAAT
EP4 Probe
ACG CGG GCT TCA GCT CCT TCC T
PDE4b primers
CCTTCAGTAGCACCGGAATCA
CAAACAAACACACAGGCATGTAGTT
PDE4b Probe
AGCCTGCAGCCGCTCCAGCC
Granulysin primers
CAGGGTGTGAAAGGCATCTCA
GGAGCATGGCTGCAAGGA
Granulysin Probe
CGGCTGCCCCACCATGGC
CD14 primers
GCGCTCCGAGATGCATGT
AGCCCAGCGAACGACAGA
CD14 Probe
TCCAGCGCCCTGAACTCCCTCA
E synthase primers
CGGAGGCCCCCAGTAflG
GGGTAGATGGTCTCCATGTCGTT
E synthase Probe
CGACCCCGACGTGGAACGCT
IRAKM primers
CCT GCC CTC GGA ATT TCT CT
CTT TGC CCG CGT TGC A
IRAKM probe
CAC ACC GGC CTG CCA AAC AGA A
CIITA primers
GCTGTTGTGTGACATGGAAGGT
RTGGGAGTCCTGGAAGACATACTG
CIITA Probe
CCGCGATATTGGCATAAGCCTCCCT
Class II primers
AGCCCAACGTCCTCATCTGT
TCGAAGCCACGTGACATTGA
Class II Probe
TCATCGACAAGTTCACCCCACCAGTG

Template was amplified in a Taqman 7700 machine for 40 cycles using FAM/TAMRA dyes on the probe. The Applied Biosystems Kit was used to amplify and detect ribosomal (18S)RNA as a control. After 40 cycles the Ct (related to cycle number at which signal appears) for the FAM and the 18S (VIC) were recorded and absolute relative quantitation was achieved using the formula 2^{-ΔΔCt}.

The results of this experiment are shown in FIG. 2 and show that there is a synergistic between a prostaglandin (PGE2) and GMCSF on the release of IL-10, CD-14, CD86, COX-2, and granulysin from cells of the immune system.

### EXAMPLE 2

### Prostaglandin E/GMCSF Synergism for Inducing IL-1

Cells were cultured as described in Example 1 but after 4 hours medium was removed, cells were washed and the cells were cultured in medium alone for a further 48 hours. RNA was extracted from the cells as described in Example 1.

The results of this experiment are shown in FIG. 3 and show that there is a synergistic effect between a prostaglandin (PGE2) and GMCSF on the expression of IL-10, and that this phenotype is maintained 48 hours after removal of the treatment.

### EXAMPLE 3

### Release of IL-10 from Monocytes in Response to PGE and GMCSF

U937 cells were grown in RPMI (PAA Laboratories) medium with 10% foetal calf serum (PAA Laboratories) added. Cells were treated with prostaglandin E2 at 10⁻⁶ Molar both with and without GMCSF at 5 ng/ml for 4 hours. The treatment was removed and cells were cultured for a further 20 hours. Medium was removed and assayed for IL-10 using a matched monoclonal antibody pair (Pharmingen) or a commercial ELISA (R&D Systems, catalogue number D1000, Abingdon, Oxford). FIG. 4 shows the release of IL-10 from monocytes in response to PGE and GMCSF.

To assay for cyclic AMP levels, wells in which cells are growing are treated with 0.01N hydrochloric acid to extract intracellular cAMP. This extract is neutralised to pH 6 and assayed for cyclic AMP in a competitive enzyme immunoassay (R&D Systems, catalogue numer DE0450, Abingdon, Oxford).

### EXAMPLE 4

### Relative Efficacy of Various Agents which Raise Camp Levels in Monocyte Cells in Inducing IL-10

Experimental Details

U937 (human monocyte cell line) cells were grown in RPMI (PAA Laboratories) medium with 10% fetal calf serum added (PAA Laboratories). Cells were treated with prostaglandin E2 at 10⁻⁶ Molar, Rolipram 10⁻⁶ Molar, Forskolin 50×10⁻⁶ Molar with or without GMCSF at 5 ng/ml for 48 hours. Cells were pelleted and the mRNA was extracted with Tri reagent (Sigma, Poole, UK). Total RNA was obtained by addition of chloroform and subsequent isopropanol precipitation. RNA was reverse transcribed with reverse transcriptase (Applied Biosystems) and random hexamers (Applied Biosystems). Probes and primers for amplification and detection of IL-10 were designed using Primer Express (Applied Biosystems) and are as follows:

IL-10 primers
CTACGGCGCTGTCATCGAT
TGGAGCTTATTAAAGGCATTCTTCA
IL-10 probe
CTTCCCTGTGAAAACAAGAGCAAGGCC

See FIG. 6.

### EXAMPLE 5

### Relative Efficacy of Various Agents which Raise cAMP Levels in Monocyte Cells in Inducing IL-10 Compared to TNFα

As for Example 4 but mRNA for TNFα alpha is also included.

PMA (2×10⁻⁷ M) was used as an alternative differentiating agent and although IL-10 was increased by PMA differentiation, TNFα (a pro-inflammatory and antitolerogenic agent) was also increased. Differentiation with Forskolin and GMCSF did not appreciably raise TNFα. Data is shown as the ratio of IL-10 mRNA/TNFα mRNA. P═PMA=Phorbol myristoyl acetate; F=Fsk=Forskolin, g=GMCSF, C=vehicle control.

TNFα Primers
GGAGAAGGGTGACCGACTCA
TGCCCAGACTCGGCAAAG
TNFα probe
CGCTGAGATCAATCGGCCCGACTA

See FIG. 7.

### EXAMPLE 6

### Relative Efficacy of Various Agents which Raise cAMP Levels in Monocyte Cells in Inducing Granulysin

As for Example 4 but mRNA for granulysin was measured using the primers listed in Example 1 (see FIG. 8).

G=GMCSF; FSK=Forskolin

### EXAMPLE 7

### Prostaglandin E/GMCSF/Probenecid Synergism for Inducing IL-10

Cells were cultured as described in Example 1 but after 20 hours medium was removed, cells were washed and RNA was extracted from the cells as described in Example 1.

The results of this experiment are shown in FIG. 9 and show that there is a synergistic effect between a prostaglandin (PGE2) and GMCSF and probenecid on the expression of IL-10.

E=PGE2

### EXAMPLE 8

### Treatment of Demyelinating Disease

A patient with demyelinating disease is administered 800 μg misoprostol orally and 250 μg/m² Leukine® intravenously day, together with 200 μg myelin.

### EXAMPLE 9

### Treatment of Rheumatoid Arthritis

A patient with rheumatoid arthritis is administered 800 μg misoprostol orally and 250 μg/m² Leukine® subcutaneously per day together with 200 μg type II collagen.

### EXAMPLE 10

A patient with rheumatoid arthritis is administered 200 μg Collagen Type II, 100 μg Forskolin and 100 μg Leukine as a suppository twice daily.

### EXAMPLE 11

A patient with rheumatoid arthritis is administered 200 μg Collagen Type II, 100 μg PGE, 4 mg probenecid and 100 μg Leukine as a suppository twice daily.

### EXAMPLE 12

A patient with rheumatoid arthritis is administered 200 μg Collagen Type II, 10 micrograms of cholera toxin, and 100 μg Leukine as a suppository twice daily.

### EXAMPLE 13

A patient with rheumatoid arthritis is administered 200 μg Collagen Type II, 100 μg 8-br cAMP and 100 μg Leukine® as a suppository twice daily.

### EXAMPLE 14

A female patient with multiple sclerosis is administered, vaginally, a gel suspension of 1 mg Myelin, 500 μg Rolipram, 100 μg PGE2, 4 mg probenecid and 100 μg Leukine.

### REFERENCES

Burzyn, D., C. C. Jancic, et al. (2002). “Decrease in cAMP levels modulates adhesion to fibronectin and immunostimulatory ability of human dendritic cells.” J Leukoc Biol 72(1): 93-100.
Harizi, H., M. Juzan, et al. (2002). “Cyclooxygenase-2-issued prostaglandin e(2) enhances the production of endogenous IL-10, which down-regulates dendritic cell functions.” J Immunol 168(5): 2255-63.
Hata, A., L. Zerboni, M. Sommer, A. A. Kaspar, C. Clayberger, A. M. Krensky and A. M. Arvin (2001). “Granulysin blocks replication of varicella-zoster virus and triggers apoptosis of infected cells.” Viral Immunol 14(2): 125-33.
Kalinski, P., C. M. Hilkens, A. Snijders, F. G. Snijdewint and M. L. Kapsenberg (1997). “Dendritic cells, obtained from peripheral blood precursors in the presence of PGE2, promote Th2 responses.” Adv Exp Med Biol 417: 363-7.
Kraan, T. C. T. M. V., L. C. M. Boeije, R. J. T. Smeenk, J. Wijdenes and L. A. Aarden (1995). “Prostaglandin E-E2 Is a potent inhibitor of human interleukin-12 production.” Journal of Experimental Medicine 181(2): 775-779.
Krensky, A. M. (2000). “Granulysin: a novel antimicrobial peptide of cytolytic T lymphocytes and natural killer cells.” Biochem Pharmacol 59(4): 317-20.
Louis, E., D. Franchimont, M. Deprez, A. Lamproye, N. Schaaf, P. Mahieu and J. Belaiche (1996). “Decrease in systemic tolerance to fed ovalbumin in indomethacin-treated mice.” International Archives of Allergy and Immunology 109(1): 21-26.
Newberry, R. D., J. S. McDonough, W. F. Stenson and R. G. Lorenz (2001). “Spontaneous and continuous cyclooxygenase-2-dependent prostaglandin(2) production by stromal cells in the murine small intestine lamina propria: directing the tone of the intestinal immune response.” J Immunol 166(7): 4465-72.
Newberry, R. D., W. F. Stenson and R. G. Lorenz (1999). “Cyclooxygenase-2-dependent arachidonic acid metabolites are essential modulators of the intestinal immune response to dietary antigen.” Nature Medicine 5(8): 900-6.
Ochoa, M. T., S. Stenger, P. A. Sieling, S. Thoma-Uszynski, S. Sabet, S. Cho, A. M. Krensky, M. Rollinghoff, E. Nunes Sarno, A. E. Burdick, T. H. Rea and R. L. Modlin (2001). “T-cell release of granulysin contributes to host defense in leprosy.” Nat Med 7(2): 174-9.
Rugtveit, J., A. Bakka and P. Brandtzaeg (1997). “Differential distribution of B7.1 (CD80) and B7.2 (CD86) costimulatory molecules on mucosal macrophage subsets in human inflammatory bowel disease (IBD).” Clin Exp Immunol 110(1): 104-13.
Scheuer, W. V., M. V. Hobbs and W. O. Weigle (1987). “Interference with tolerance induction in vivo by inhibitors of prostaglandin synthesis.” Cellular Immunology 104(2): 409-418.
Schwacha, M. G., C. S. Chung, et al. (2002). “Cyclooxygenase 2-mediated suppression of macrophage interleukin-12 production after thermal injury.” Am J Physiol Cell Physiol 282(2): C263-70.
Smyth, M. J., J. M. Kelly, V. R. Sutton, J. E. Davis, K. A. Browne, T. J. Sayers and J. A. Trapani (2001). “Unlocking the secrets of cytotoxic granule proteins.” J Leukoc Biol 70(1): 18-29.
Strassmann, G., V. Patil Koota, F. Finkelman, M. Fong and T. Kambayashi (1994). “Evidence for the involvement of interleukin 10 in the differential deactivation of murine peritoneal macrophages by prostaglandin2.” Journal of Experimental Medicine 180(6): 2365-70.

1. A method of inducing tolerance to an antigen in a patient, the method comprising administering to the patient an agent which raises the effective cAMP concentration in a monocyte cell and granulocyte-macrophage colony stimulating factor (GMCSF) or a derivative thereof.

2. A method according to claim 1 wherein the agent which raises the effective cAMP concentration in a monocyte cell is any one or more of a prostaglandin or agonist thereof, a β-adrenergic agent, a blocker of cAMP export from the cell, forskolin or a derivative thereof, a cAMP phosphodiesterase inhibitor, a cAMP analogue, or cholera toxin or a derivative or fragment thereof.

3. A method according to claim 2 wherein the blocker of cAMP export from the cell is probenicid or progesterone.

4. A method according to claim 2 wherein the cAMP analogue is Sp-adenosine 3′,5′-cyclic monophosphorothioate or 8-bromoadenosine 3′,5′ cyclic monophosphate or dibutryryl cAMP.

5. A method according to claim 2 wherein the prostaglandin or agonist thereof stimulates cAMP production in a monocyte.

6. A method according to claim 2 wherein the prostaglandin or agonist thereof is any one of a prostaglandin E or an analogue thereof, prostaglandin E₂ or an analogue thereof, dinoprostone, gemeprost, misoprostol, alprostadil, limaprost, butaprost, 11-deoxy PGE1, AH23848, AH13205, or a 19-hydroxy PGE.

7. A method according to claim 1 wherein the GMCSF is human GMCSF having the amino acid sequence of SEQ ID NO:2 or naturally occurring variants thereof.

8. A method according to claim 1 wherein the GMCSF is sargramostim.

9. A method according to claim 1 further comprising administering to the patient one or more of a monocyte chemotactic agent, a phosphodiesterase (PDE) inhibitor, and the antigen or a derivative thereof.

10. A method according to claim 9 wherein the monocyte chemotactic agent is MCP-1 or MIP-1α.

11. (canceled)

12. A method according to claim 9 wherein the PDE inhibitor is any one of 3-isobutyl-1-methylxanthine (IBMX), pentoxifylline (3,7-dihydro-3,7-dimethyl-1-(5-oxohexyl)-1H-purine-2,6-dione), rolipram (4-[3-cyclopentyloxy-4-methoxyphenyl]-2-pyrrolidinone), CP80 633, CP102 995, CP76 593, Ro-20-1724 (4-[3-butoxy-4-methoxybenzyl]-2-imidazolidinone), theophylline, or denbufylline (1,3-di-n-butyl-7-(2-oxopropyl)-xanthine).

13. A method according to claim 9 wherein the PDE inhibitor is selective for type IV PDE.

14. A method according to claim 13 wherein the PDE inhibitor selective for type IV PDE is any one of rolipram (4-[3-cyclopentyloxy-4-methoxyphenyl]-2-pyrrolidinone), CP80 633, CP102 995, CP76 593, Ro-20-1724 (4-[3-butoxy-4-methoxybenzyl]-2-imidazolidinone), denbufylline (1,3-di-n-butyl-7-(2-oxopropyl)-xanthine), or CDP840, RP73401 or RS33793.

15. (canceled)

16. A method according to claim 9 wherein one or more of the agent which raises the effective cAMP concentration in a monocyte cell, the GMCSF or derivative thereof, the monocyte chemotactic agent, the PDE inhibitors and the antigen or derivative thereof is administered (i) locally at a site where tolerance is required, (ii) systemically, (iii) orally, or (iv) as a suppository or capsule.

17-19. (canceled)

20. A method according to claim 16 wherein the suppository or capsule has an enteric coating for release of the one or more of the agent which raises the effective cAMP concentration in a monocyte cell, the GMCSF or derivative thereof, the monocyte chemotactic agent, the PDE inhibitor, and the antigen or derivative thereof in the bowel of the patient.

21. A method according to claim 16 wherein at least the GMCSF or derivative thereof is administered subcutaneously or intravenously.

22. A method according to claim 9 wherein any two or more of the agent which raises the effective cAMP concentration in a monocyte cell, the GMCSF or derivative thereof, the monocyte chemotactic agent, the PDE inhibitors and the antigen or derivative thereof are administered simultaneously.

23. A method of combating a disease or condition associated with transplant rejection comprising:
performing the method according to claim 1, wherein said administering is effective to combat a disease or condition associated with transplant rejection.

24. A method according to claim 23 wherein the disease or condition associated with transplant rejection comprises graft versus host disease or host versus graft disease.

25. A method according to claim 23 wherein one or more of the agent which raises the effective cAMP concentration in a monocyte cell, the GMCSF or derivative thereof, a monocyte chemotactic agent, a PDE inhibitor, and the antigen or derivative thereof is administered prior to the transplant.

26. A method according to claim 23 wherein the antigen is HLA-A2.

27. A method of treating an autoimmune disease or condition comprising:
performing the method according to claim 1, wherein said administering is effective to treat an autoimmune disease or condition.

28. A method according to claim 27 wherein the autoimmune disease is selected from the group consisting of primary myxoedema, thyrotoxicosis, pernicious anaemia, autoimmune atrophic gastris, Addison's disease, insulin-dependent diabetes mellitus (IDDM), Goodpasture's syndrome, myasthenia gravis, sympathetic ophthalmia, multiple sclerosis (MS), autoimmune haemolytic anaemia, idiopathic leucopenia, ulcerative colitis, dermatomyositis, scleroderma, mixed connective tissue disease, rheumatoid arthritis, irritable bowel syndrome, systemic lupus erythromatosus (SLE), Hashimoto's disease, thyroiditis, Behcet's disease, coeliac disease/dermatitis herpetiformis, renal vasculitis, and demyelinating disease.

29. A method according to claim 27 wherein the antigen is a self-antigen.

30. A method according to claim 27, wherein the autoimmune disease is pernicious anaemia, and the antigen is vitamin B₁₂; the disease is Addison's disease, and the antigen is adrenal antigen; the disease is IDDM, and the antigen is glutamic acid decarboxylase (GAD), insulin, or IA-2; the disease is Goodpasture's syndrome or renal vasculitis, and the antigen is renal antigen or endothelial antigen; the disease is myasthenia gravis, and the antigen is the acetyl choline receptor; the disease is sympathetic ophthalmia, and the antigen is ocular antigen; the disease is multiple sclerosis (MS), and the antigen is myelin basic protein (MBP), proteolipid protein (PLP), or myelin oligodendrocyte glycoprotein (MOG); the disease is autoimmune haemolytic anaemia, and the antigen is red cell antigen; the disease is idiopathic leucopenia, and the antigen is leukocyte antigen; the disease is ulcerative colitis, and the antigen is a food antigen or a viral antigen; the disease is dermatomyositis, and the antigen is smooth muscle antigen; the disease is scleroderma, and the antigen is a connective tissue antigen; the disease is mixed connective tissue disease, and the antigen is a connective tissue antigen; the disease is irritable bowel syndrome, and the antigen is a food antigen; the disease is systemic lupus erythmatosus (SLE), and the antigen is a histone protein or immunoglobulin heavy chain; the disease is Hashimoto's disease, primary myxoedema or thyrotoxicosis, and the antigen is thyroid antigen; the disease is rheumatoid arthritis, and the antigen is type II collagen or a heat shock protein (HSP); the disease is thyroiditis, and the antigen is thyroglobulin; the disease is Behcet's disease, and the antigen is Sag, HLA-B44, B51, or HSP65; the disease is Coeliac disease/Dermatitis herpetiformis, and the antigen is gliadin or the α fraction thereof; or the disease is demyelinating disease, and the antigen is myelin.

31. A method of treating an allergic disease or condition in a patient comprising:
performing the method according to claim 1, wherein said administering is effective to treat an allergic disease or condition in the patient.

32. A method according to claim 31 wherein the allergic disease or condition is allergic asthma.

33. A method according to claim 31, wherein the antigen is a mite allergen, a dust allergen, a cat allergen, a dog allergen or a horse allergen.

34. A method according to claim 1, wherein the induced tolerance to the antigen is effective to treat an aberrant or undesired immune or inflammatory response to the antigen in the patient.

35. A method according to claim 34 wherein the aberrant or undesired immune or inflammatory response involves a deficiency in IL-10 production.

36. A composition comprising an agent which raises the effective cAMP concentration in a monocyte cell and granulocyte-macrophage colony stimulating factor (GMCSF) or a derivative thereof.

37. A composition according to claim 36 wherein the agent which raises the effective cAMP concentration in a monocyte cell is any one or more of a prostaglandin or agonist thereof, a β-adrenergic agent, a blocker of cAMP export from the cell, forskolin or a derivative thereof, a cAMP phosphodiesterase inhibitor, a cAMP analogue, or cholera toxin or a derivative or fragment thereof.

38. A composition according to claim 37 wherein the blocker of cAMP export from the cell is probenicid or progesterone.

39. A composition according to claim 37 wherein the cAMP analogue is Sp-adenosine 3′,5′-cyclic monophosphorothioate or 8-bromoadenosine 3′,5′ cyclic monophosphate.

40. A composition according to claim 37 wherein the prostaglandin or agonist thereof stimulates cAMP production in a monocyte.

41. A composition according to claim 37 wherein the prostaglandin or agonist thereof is a prostaglandin E or an analogue thereof, prostaglandin E₂ or an analogue thereof, dinoprostone, gemeprost, misoprostol, alprostadil, limaprost, butaprost, 11-deoxy PGE1, AH23848, AH13205, or a 19-hydroxy PGE.

42. A composition according to claim 36 wherein the GMCSF is human GMCSF having the amino acid sequence of SEQ ID NO:2 or naturally occurring variants thereof.

43. A composition according to claim 36 wherein the GMCSF is sargramostim.

44. A composition according to claim 36 further comprising one or more of a monocyte chemotactic agent, a phosphodiesterase (PDE) inhibitor, and an antigen or derivative thereof.

45. A composition according to claim 44 wherein the monocyte chemotactic agent is MCP-1 or MIP-1α.

46. (canceled)

47. A composition according to claim 44 wherein the PDE inhibitor is any one of 3-isobutyl-1-methylxanthine (IBMX), pentoxifylline (3,7-dihydro-3,7-dimethyl-1-(5-oxohexyl)-1H-purine-2,6-dione), rolipram (4-[3-cyclopentyloxy-4-methoxyphenyl]-2-pyrrolidinone), CP80 633, CP102 995, CP76 593, Ro-20-1724 (4-[3-butoxy-4-methoxybenzyl]-2-imidazolidinone), theophylline, or denbufylline (1,3-di-n-butyl-7-(2-oxopropyl)-xanthine).

48. A composition according to claim 44 wherein the PDE inhibitor is selective for type IV PDE.

49. A composition according to claim 48 wherein the PDE inhibitor selective for type IV PDE is any one of rolipram (4-[3-cyclopentyloxy-4-methoxyphenyl]-2-pyrrolidinone), CP80 633, CP102 995, CP76 593, Ro-20-1724 (4-[3-butoxy-4-methoxybenzyl]-2-imidazolidinone), denbufylline (1,3-di-n-butyl-7-(2-oxopropyl)-xanthine), or CDP840, RP73401 or RS33793.

50. (canceled)

51. A pharmaceutical composition comprising the composition according to claim 36 and a pharmaceutically acceptable carrier, diluent or excipient.

52-65. (canceled)

66. A therapeutic system for inducing tolerance to an antigen in a patient, the system comprising an agent which raises the effective cAMP concentration in a monocyte cell and GMCSF or a derivative thereof.

67. A therapeutic system according to claim 66 further comprising one or more of the antigen, a monocyte chemotactic agent, and a phosphodiesterase (PDE) inhibitor.

68. (canceled)

69. A therapeutic system according to claim 67 wherein one or more of the agent which raises the effective cAMP concentration in a monocyte cell, the GMCSF or derivative thereof, the monocyte chemotactic agent, the PDE inhibitor, and the antigen or derivative thereof is in a preparation for (i) administration locally at a site where tolerance is required, (ii) systemic administration, (iii) oral administration, or (iv) administration as a suppository or capsule.

70-72. (canceled)

73. A method of stimulating or enhancing granulysin expression in cells of a macrophage/monocyte lineage comprising administering to the cells a therapeutic system according to claim 66.

74. A method of treating a viral infection in a patient comprising administering to the patient a therapeutic system according to claim 66.

75. A method according to claim 74 wherein the viral infection is a herpes simplex virus infection or a human papilloma virus infection.

76-77. (canceled)

78. A method of stimulating or enhancing IL-10 expression in, and secretion from, cells of a macrophage/monocyte lineage comprising administering to the cells a therapeutic system according to claim 66.

79. A method of treating a tumour in a patient comprising administering to the patient a therapeutic system according to claim 66.

80-81. (canceled)
